(19) United States
(12) Patent Application Publication
(10) Pub. No.: US 2010/0104511 A1
(43) Pub. Date: Apr. 29, 2010

(54) METHODS AND COMPOSITIONS USING CHELATOR-ANTIBODY CONJUGATES

(75) Inventors: Razelle Kurzrock, Bellaire, TX (US); Jing Gong, Manvel, TX (US); David Yang, Sugar Land, TX (US); Saady Kohanim, Sugar Land, TX (US)

Correspondence Address: FULBRIGHT & JAWORSKI L.L.P., 600 CONGRESS AVE., SUITE 2400, AUSTIN, TX, 78701, US

(73) Assignee: The Board of Regents of the University of Texas System, Austin, TX (US)

(21) Appl. No.: 12/516,172

(22) PCT Filed: Nov. 14, 2007
(86) PCT No.: PCT/US07/84694

§ 371 Date: Dec. 15, 2009

Related U.S. Application Data
(60) Provisional application No. 60/867,008, filed on Nov. 22, 2006.

(51) Int. Cl. A61K 51/00; C07K 16/00; A61P 35/04; A61P 29/00

(52) U.S. Cl. 424/1.53; 530/391.1; 530/391.3

(57) ABSTRACT

Disclosed are pharmaceutical compositions that include a chelator and an antibody directed against a phosphorylation site of a protein, wherein the antibody is conjugated to the chelator to form a chelator-antibody conjugate. For example, the antibody may recognize a phosphorylated tyrosine residue or a phosphorylated serine residue of a cell surface receptor. Also disclosed are methods of synthesizing a radiolabeled chelator-antibody conjugate, wherein the antibody is directed against a phosphorylation site of a protein. Also disclosed are methods for imaging a site in a subject that involve administering to the subject an effective amount of a composition that includes a valent metal ion-labeled chelator-antibody conjugate, wherein the antibody is an antibody directed against a phosphorylation site of a protein, and detecting a radioactive signal from the site in the subject following administration of an effective amount of the composition. The method of imaging can be applied in diagnosing a tumor, such as a tumor that can be responsive to therapy using a tyrosine kinase inhibitor, and in the treatment of a tumor, such as by targeting therapy to cell surface receptors that include phosphorylation sites.

This application claims the benefit of priority to U.S. Provisional Patent Application Ser. No. 60/867,008, filed Nov. 22, 2006, the entire contents of which is hereby incorporated by reference in its entirety.

### BACKGROUND OF THE INVENTION

1. Field of the Invention

The present invention relates generally to the fields of labeling, radioimaging, chemical synthesis, and clinical oncology. More particularly, it concerns radiolabeled chelator-antibody conjugates and methods of synthesis of radiolabeled chelator-antibody conjugates, wherein the antibody is an antibody directed against a phosphorylation site of a protein. It also concerns methods of imaging and therapy using radiolabled chelator-antibody conjugates, including a method of predicting response of a patient to phosphotyrosine therapy.

2. Description of Related Art

Cancer is the second most common cause of death in developed countries and is a rising health problem in less developed parts of the world. For many patients, conventional therapies (surgery, radiation therapy, and chemotherapy) have a high toxicity and marginal efficacy. Thus, there is great interest in the identification of new forms of therapy.

One relatively new form of anti-cancer therapy is therapy using tyrosine kinase inhibitors (reviewed in Tibes et al., 2005). A tyrosine kinase catalyzes the phosphorylation of a tyrosine residue to form a phosphorylated tyrosine residue in a protein (phospho-tyrosine). Phosphorylation of tyrosine residues by tyrosine kinase is involved in cellular processes including the cell cycle, migration, metabolism, proliferation, survival and differentiation of cells. Examples of tyrosine kinases include epidermal growth factor receptor (EGFR), Bcr-Abl, KIT, platelet-derived growth factor receptor (PDGFR), and vascular endothelial growth factor (VEGF).

Epidermal growth factor receptor (EGFR) is a membrane-bound receptor tyrosine kinase expressed in a variety of human solid tumors (Boonstra et al., 1995; Mendelsohn and Baselga, 2000). Upon ligand binding, the receptor forms homo- or heterodimers leading to autophospholation of key tyrosine residues in the cytosolic domains of the proteins (Karunagaran et al., 1996; Graus-Porta et al., 1997). This process initiates receptor-mediated signal transduction that effects cell proliferation and survival.

The blockade of EGFR with gefitinib, a small molecule EGFR tryosine kinase inhibitor, has been shown to have marked antiproliferative effects against tumors in culture (Sato et al., 1983; Sarup et al., 1991) and in animals (Masui et al., 1984; Park et al., 1991). Further studies have shown that the presence of activating EGFR mutations in lung cancer correlate well with clinical response to gefitinib therapy (Paez et al., 2004).

Methods to assess which patients would benefit from a specific therapy or methods to assess the efficacy of anti-cancer therapies are limited. To assess the efficacy of anti-cancer therapy using tyrosine kinase inhibitors or to assess which patients would benefit from tyrosine kinase inhibitor therapy, it would be important to measure phospho-tyrosine activity after treatment. No clinically useful method is known in the art.

PET and SPECT use radiotracers to image, map and measure target site activities (e.g., angiogenesis, metabolism, apoptosis and proliferation) and they are considered as targeted molecular imaging modalities (Yang and Kim, 2005). To assess clinical endpoints of tyrosine kinase inhibitor therapy, a specific target assessment marker is needed that would allow precise measurement of tumor targets on a whole-body image upon administration of a functional agent. Reliable molecular imaging agents assess treatment response more rapidly, and predict therapeutic response would be extremely valuable in of itself. In addition, such agents, if linked to a radio ablative molecule could be therapeutic.

To develop novel or clinically used tracers, two types of chemistries are frequently used in the preparation of radiotracers: covalent and ionic. In covalent chemistry, either displacement or addition reactions are used to place an isotope in the molecule. The labeled product provides minimal structural alteration, however, the procedure may be lengthy, tedious, with low yield, and costly. Isotopes commonly used in covalent chemistry include ¹⁸F, ¹²³I, ¹³¹I, ⁷⁵Br, ⁷⁷Br and ¹¹C. In complexation chemistry, a chelator is required to trap metal isotopes. This type of chemistry is simple and with high yield. The isotopes may be obtained from generators. Though complexation chemistry is attractive, the chemical properties may be altered due to the addition of a chelator.

Several chelators have been reported, such as N₄ (e.g., DOTA), N₃S (e.g., MAG-3), N₂S₂ (e.g., ECD), NS₃, S₄ (e.g., sulfur colloid), diethylenetriamine pentaacetic acid (DTPA), and O₂S₂ (e.g., DMSA) (Van Nerom et al., 1993; Laissy et al., 1994; Wu et al., 2003). Among these chelators, the nitrogen and sulfur combination has been shown to be a stable chelator. L,L-ethylenedicysteine (EC) is the most successful example of an N₂S₂ chelate. EC can be labeled with metallic isotopes efficiently with high radiochemical purity and the preparation remains stable for several hours (Yang et al., 2005). It has been previously reported that a series of EC-agent conjugates could target the tumor targets (Yang et al., 2001; Yang et al., 2005; Yang et al., 2002; Yang et al., 2004a; Schechter et al., 2003; Song et al., 2003; Yang et al., 2003; Yang et al., 2004b).

### SUMMARY OF THE INVENTION

The inventors have identified certain novel chemical conjugates that can be applied in predicting which patients would benefit from a particular therapy. The conjugates include a chelator conjugated to an antibody directed against a phosphorylation site of a protein. For example, the inventors developed a novel radio labeled anti-phospho-tyrosine antibody to assess phospho-tyrosine activity in patients with a tumor. They have found that the anti-phospho-tyrosine activity of tyrosine kinase inhibitors such as gefitinib can be measured by in vivo imaging using the radiolabeled chelator-antibody conjugates, such as indium-labeled phospho-tyrosine antibody (¹¹¹In-EC-P-Tyr). Further, they have found that down-regulation of phospho-tyrosine correlates with anti-tumor responses. Thus, the radiolabeled chelator-antibody conjugates of the present invention can be applied as a noninvasive functional imaging technique to select potential responsive vs resistant patients based on baseline expression. Further, imaging using these conjugates can be applied in determining therapeutic efficacy following a course of therapy that would be beneficial to patients early on in the course of treatment.

Embodiments of the present invention generally concern pharmaceutical compositions that include (1) a chelator; and (2) an antibody directed against a phosphorylation site of a protein, wherein the antibody is conjugated to the chelator to form a chelator-antibody conjugate.

The antibody can be any antibody that is directed to a phosphorylation site of a protein. In particular embodiments, the protein is a protein that is a receptor. In some embodiments, the receptor is a cell surface receptor. The cell surface receptor can be any cell surface receptor that includes a phosphorylation site. For example, in particular embodiments, the cell surface receptor is a growth factor receptor.

The antibody, for example, may recognize a phosphorylated tyrosine residue (phosphotyrosine antibody) or a phosphorylated serine residue (phosphoserine antibody). For example, the antibody may be directed against any of those tyrosine kinases set forth in FIG. 1-FIG. 3. The antibody may recognize a protein phosphorylation site of a receptor on the outer surface of a cell membrane. Alternatively the antibody may recognize a protein phosphorylation site of a receptor on the inner surface of a cell membrane.

In particular examples, the antibody is directed against a phosphorylated epidermal growth factor receptor (phospho-EGFR antibody), a phorphorylated platelet derived growth factor receptor (phospho-PDGFR antibody), a phosphorylated KIT (phospho-KIT antibody), or a phosphorylated Bcr-Abl antibody (phospho-Bcr-Abl antibody).

Any method known to those of ordinary skill in the art can be applied in preparing an antibody directed against a phosphorylation site of a protein. Information and examples pertaining to such methods are discussed in the specification below.

A “chelator” is defined herein to refer to a compound that comprises one or more atoms that are capable of chelating one or more valent metal ions. Persons of skill in the art will be familiar with compounds that are considered to be chelators. Chelators comprising three or four atoms available for chelation are used as chelators in particular embodiments of the present chelator-antibody conjugates. In a further particular embodiment, the chelator chelates to one valent metal ion. In some embodiments, the atoms available for chelation are selected from the group consisting of nitrogen, sulfur, oxygen, and phosphorus. For example, the chelator may be selected from the group consisting of an NS₂ chelator, an N₂S chelator, an N₄ chelator, an S₄ chelator, an N₂S₂ chelator, an N₃S chelator, and an NS₃ chelator. In particular embodiments, the chelator is an N₂S₂ chelator.

In particular embodiments, the chelator is a bis-aminoethanethiol dicarboxylic acid. For example, the bis-aminoethanethiol dicarboxylic acid may be N,N-ethylenedicysteine (EC). EC and analogs of EC are discussed in greater detail in the specification below.

Any method of conjugating the chelator to the antibody that is known to those of ordinary skill in the art is contemplated by the present invention. Examples of methods and techniques that can be applied are discussed in greater detail in the specification below. For example, the chelator may be conjugated to the amino terminus of the antibody or a lysine residue of the antibody.

In particular embodiments, the pharmaceutical composition includes a valent metal ion chelated to the chelator-antibody conjugate. Any valent metal ion known to those of ordinary skill in the art is contemplated by the present invention.

In particular embodiments, the valent metal ion is a radionuclide. For example, the radionuclide may be a radionuclide selected from the group consisting of Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90. More particularly, the valent metal ion may be is In-111.

In some embodiments, the pharmaceutical composition includes two or more valent metal ions chelated to the chelator-antibody conjugate. The two or more valent metal ions may or may not be identical. In some embodiments, one of the valent metal ions is a therapeutic valent metal ion, such as a beta emitter. For example, the beta emitter may be Re-188, Re-186, Ho-166, Y-90, and Sn-153. The two or more valent metal ions may be chelated to the chelator, the antibody, or both chelator and antibody. In particular embodiments, the pharmaceutical composition includes In-111 and U-90.

In some embodiments set forth herein, the valent metal ion is chelated only to the chelator. In other embodiments, the valent metal ion is chelated only to the antibody. For example, the valent metal ion may be chelated to a carboxylic acid moiety of a glutamate or aspartate residue of the antibody. In other embodiments, the valent metal ion is chelated to both the chelator and the antibody. Methods of chelation are discussed at length in the specification below.

In particular embodiments, the chelator is EC and the antibody is an antibody directed against a phosphorylated tyrosine residue of a protein (phosphotyrosine antibody).

The present invention also generally pertains to methods of synthesizing a radiolabeled chelator-antibody conjugate that includes (1) obtaining an antibody directed against a phosphorylation site of a protein; (2) admixing the antibody with a chelator to obtain a chelator-antibody conjugate; and (3) admixing the chelator-antibody conjugate with a radionuclide to obtain a radionuclide labeled chelator-antibody conjugate. The antibody directed against a phosphorylation site of a protein can be any of the antibodies discussed above. In particular embodiments, the antibody is a phosphotyrosine antibody or a phosphoserine antibody. The chelator can be any of those chelators discussed above and elsewhere in this specification. In certain embodiments, the chelator is a bis-aminoethanethiol dicarboxylic acid. In particular embodiments, the chelator is EC. The radionuclide can be any of the radionuclides set forth above. In particular embodiments, the radionuclide is In-111.

Admixing the chelator-antibody conjugate with the radionuclide can be by any method known to those of ordinary skill in the art. In particular embodiments, admixing the chelator-antibody conjugate with the radionuclide is performed in an aqueous media. The aqueous media may include one or more additional components. For example, in some embodiments, the aqueous media includes carbodiimide and sulfo-N-hydroxysuccinimide. In some embodiments, the chelator-antibody conjugate is admixing with a radionuclide in the presence of a reducing agent. The reducing agent can be any reducing agent known to those of ordinary skill in the art. For example, the reducing agent may be stannous chloride (SnCl₂), dithionate ion, or ferrous ion. Information regarding chelation of a valent metal ion to a conjugate is discussed in greater detail below.

The present invention also generally pertains to methods of imaging a site in a subject. These methods generally involve (1) administering to the subject an effective amount of a first composition that includes a valent metal ion-labeled chelator-antibody conjugate, wherein the antibody is an antibody directed against a phosphorylation site of a protein; and (2) detecting a radioactive signal from the site in the subject following administration of an effective amount of the first composition.

The term “subject” refers to either a human or non-human, such as primates, mammals, and vertebrates. In particular embodiments, the subject is a human. In more particular embodiments, the human is a patient with a disease.

In some embodiments, the disease is a disease associated with abnormal cell surface receptor activity. For example, the disease may be a disease associated with an alteration of tyrosine kinase activity. More particularly, the disease may be one that is associated with increased tyrosine kinase activity or increased tyrosine phosphatase activity. In particular embodiments, the disease is associated with increased tyrosine kinase activity For example, the disease associated with activation of a kinase may be a disease selected from the group consisting of cancer, an inflammatory disease, a genetic disease, an autoimmune disease, hypereosinophilic syndrome, anemia, osteoclast disease, restenosis, diabetes, and mast cell disease.

In particular embodiments, the disease is cancer. The cancer may be any type of cancer, such as breast cancer, lung cancer, prostate cancer, ovarian cancer, brain cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, leukemia, lymphoma, or sarcoma. In some embodiments, the cancer is a metastatic cancer. The cancer may be a cancer that is associated with an anborm

The cancer may be a cancer that expresses or overexpresses phospho-tyrosine residues. In other embodiments, the cancer is a cancer that expresses or overexpresses phosphoserine residues. In still further embodiments, the cancer is a cancer that expresses or overexpresses phosphotyrosine residues and phosphoserine residues. Overexpression is determined by any method known to those of ordinary skill in the art. For example, overexpression can be determined by comparing expression of phospho-tyrosine levels to other tumors, or to healthy tissue.

In some embodiments, the patient has a disease that is an inflammatory disease. For example, the inflammatory disease may be hepatitis or chronic thyroiditis. The disease may be an autoimmune disease, such as rheumatoid arthritis, systemic lupus erythematosus, or multiple sclerosis. In further embodiments, the disease is a genetic disease.

The site to be imaged is any site within a subject. For example, the site may be a site that is known or suspected of being affected by a disease. The disease, for example, can be any of those diseases set forth above. In particular embodiments, the site to be imaged is affected by a disease associated with an alteration of tyrosine kinase or tyrosine phosphatase activity. For example, the site to be imaged may be a site of a tumor, wherein the tumor expresses cell surface receptors that demonstrate increased phosphotyrosine expression or increased phosphoserine expression. The increase in phosphotyrosine expression or increase in phosphoserine expression is defined as an increase in phosphotyrosine expression or an increase in phosphoserine expression relative to the expression from a corresponding site in a healthy subject or relative to an adjacent site in the same subject.

The antibody and chelator can be any of those antibodies and chelators discussed above. In particular embodiments, the antibody is an antibody that recognizes a phosphorylated tyrosine residue or a phosphorylated serine residue. In more particular embodiments, the antibody is a phospho-EGFR antibody, a phospho-PDGFR antibody, a phospho-KIT antibody, a phospho-Bcr-Abl antibody, a phospho-VEGFR antibody, or a phospho-insulin receptor antibody.

The chelator can be any chelator discussed above. In particular embodiments, the chelator is a bis-aminoethanethiol dicarboxylic acid, such as EC. In some embodiments, the chelator is conjugated to the amino terminus of the antibody or a lysine residue of the antibody.

The valent metal ion can be any of the valent metal ions discussed above. In particular embodiments, the valent metal ion is In-111 or Y-90.

In a specific embodiments, the chelator is EC and the antibody is a phosphotyrosine antibody.

Administering an effective amount of the composition can be by any method known to those of ordinary skill in the art. For example, administering may involve intravenous, intracardiac, intradermal, intralesional, intrathecal, intracranial, intrapericardial, intraumbilical, intraocular, intraarterial, intraperitoneal, intratumor, subcutaneous, intramuscular, or intravitreous administration. In specific embodiments, administration is intravenous.

Any method known to those of ordinary skill in the art can be applied in detecting a radioactive signal from a site in a subject. For example, the signal may be detected using a signal selected from the group consisting of PET, SPECT, and gamma camera imaging. The signal that is detected may be generated into an image using any technology known to those of ordinary skill in the art.

In particular embodiments, the method of imaging a site in a subject is further defined as a method for diagnosing the presence of a disease in a subject. The disease can be any of those diseases discussed above. In particular embodiments, the disease is a cancer. For example, the disease may be a cancer that expresses cell surface receptors that demonstrate in increase in phosphotyrosine moieties or phosphoserine moieties. The phosphotyrosine moieties may have been phosphorylated by a tyrosine kinase. For example, the cell surface receptor may be EGFR, or KIT. Thus, for example, the presence of a detectable signal from a site in a subject following administration of EC-phosphotyrosine antibody may be indicative of the presence of a tumor, such as a primary tumor, that overexpresses phosphotyrosine.

The subject may be any subject, such as a subject that is suspected of having a tumor or a subject with a history of a tumor that was successfully treated with a therapy. In some embodiments, the subject has a tumor at one site, and imaging of a different site in the subject is being performed to evaluate the subject for metastatic disease. Thus, certain embodiments of the methods of imaging set forth herein are directed to methods of screening a subject for the presence of metastatic disease.

The signal that is detected is compared to a reference signal from another site in the same subject that is known to be free of disease. Alternatively, the signal can be compared to a reference signal generated from a corresponding site in a healthy subject. Alternatively, the signal can be compared to a reference signal from the site of a tumor in a second patient. For example, the tumor in the second patient may be one that is known to not express an increase in phosphotyrosine compared to normal tissue. An increase in radioactive signal from the site compared to a reference signal is indicative of the presence if disease. A “healthy subject” is defined herein to refer to a subject who is not affected by a disease.

In certain embodiments, the method of imaging is further defined as a method of determining optimal therapy in a patient with a disease. The disease, for example, can be any of those diseases discussed above. In particular examples, the disease is cancer, and the site includes a tumor. The method of determining optimal therapy in a patient with a disease such as a tumor may further involve administering to the patient an effective amount of a second valent-metal ion-labeled chelator-antibody conjugate, wherein the antibody in the second conjugate is an antibody directed against a phosphorylated site in a protein that is distinct from the antibody in the first chelator-antibody conjugate.

In some embodiments, the patient is administered a single composition that includes more than one valent metal ion-labeled chelator-antibody conjugate. In other embodiments, the patient is administered an effective amount of separate compositions of valent metal ion-labeled chelator-antibody conjugates.

In some embodiments, a single session of imaging is performed following administration of the more than one radionuclide-labeled chelator antibody conjugates. In some embodiments, more than one imaging modality is performed following administrations of the more than one radionuclide-labeled chelator antibody conjugates. In other embodiments, one or more than one imaging techniques is performed following administration of each radionuclide-labeled chelator antibody conjugate.

Thus, for example, an increase in detectable signal that is detected following administration of a particular radionuclide-labeled chelator-antibody conjugate might suggest a particular tumor responsive to a particular therapeutic modality. For example, an increase in radioactive signal following administration of a radionuclide-labeled chelator-phosphotyrosine antibody conjugate compared to a radioactive signal that is measured following administration of a radionuclide-labeled chelator-phosphoserine antibody conjugate would be indicative of the presence of a disease, such as a tumor, that would be more responsive to phosphotyrosine therapy compared to phosphoserine therapy. The antibodies that are administered to the subject in the conjugates may, for example, be selected from the group consisting of phospho-EGFR antibody, phospho-PDGFR antibody, phospho-Bcr-Abl antibody, phospho-KIT antibody, and phospho-VEGFR antibody. Thus, the methods of the present invention can be applied in determining optimum therapy of a site in a patient.

In particular embodiments, the method of imaging a site in a subject is further defined as a method for predicting a clinical response of a site in a subject to a therapy. In some embodiments, for example, the site is a tumor, and the therapy is an anticancer therapy. Examples of anticancer therapy include chemotherapy, radiation therapy, surgical therapy, gene therapy, and immune therapy. In particular embodiments, the chemotherapy is phosphotyrosine therapy. The phosphotyrosine therapy may be, for example, therapy with gefitinib, imatinib mesylate, HER-2 antibody, tiludronate, a PDGFR inhibitor, or a glucocorticoid.

Following therapy, repeat administration of an effective amount of the composition comprising a valent metal ion-labeled chelator-antibody conjugate of the present invention is performed. The site is then evaluated for the presence of a radioactive signal. A radioactive signal is detected from the site in the subject by any method known to those of ordinary skill in the art, as set forth above. The radioactive signal from the site that is detected is then compared to a radioactive signal that is detected from the site prior to a course of phosphotyrosine therapy. Thus, for example, a change in a radioactive signal from the site following therapy compared to the signal from the site prior to therapy may be indicative of a response to therapy. The change in the radioactive signal that is indicative of a response to therapy may be a decrease in the intensity of the signal, and/or a decrease in the size of an area of increased signal. An increase in the intensity of the radioactive signal and/or an increase in the size of a signal following therapy would be indicative of an increase in tumor malignancy and/or size. In some embodiments, repeat imaging is performed following administration of a second course of therapy.

In some embodiments of the present invention, the method of imaging is further defined as a method of performing dual imaging and radiochemotherapy. For example, patient may be administered a chelator-antibody conjugate that is labeled with a radionuclide suitable for imaging, and a second radionuclide suitable for radiochemotherapy. In other embodiments, the patient is administered a first valent metal ion-labeled chelator antibody conjugate that is labeled with a radionuclide suitable for imaging, and a second valent metal ion-labeled chelator antibody conjugate that is labeled with a therapeutic metal ion that may or may not be suitable for imaging. The conjugates may or may not be administered concurrently, such as in a single composition. Any method of administration known to those of ordinary skill in the art can be followed. Examples of such methods are discussed in greater detail below. The valent metal ion can be any valent metal ion, such as one of the radionuclides set forth above. In particular embodiments, the composition includes Y-90 and In-111.

The present invention also pertains to methods of targeted chemotherapy to a subject with a tumor. For example, the method may involve administering to the subject an effective amount of a composition that includes a valent metal ion-labeled antibody conjugate as set forth herein, wherein the valent metal ion is a therapeutic metal ion as set forth above and elsewhere in this specification. In some embodiments, the method further comprises imaging the tumor using any of the methods set forth herein.

The present invention also includes kits for preparing a radiopharmaceutical preparation. The kit includes one or more sealed containers, and a predetermined quantity of a chelator-antibody conjugate composition, wherein the antibody is an antibody directed against a phosphorylated site of a protein. The antibody and chelator can be any of those that have been set forth above. In particular embodiments, the chelator is EC, and the antibody is a phospho-EGFR antibody, a phospho-PDGFR antibody, a phospho-KIT antibody, a phospho-Bcr-Abl antibody, or a phospho-VEGFR antibody.

Reagents for preparing a scintigraphic imaging agent or a chemotherapeutic agent are also encompassed by the present invention. For example, the reagent may include an antibody directed against a phosphorylated site of a protein, wherein the antibody is covalently linked to a chelator. The antibody and chelator can be any of those antibodies and chelators discussed above. The antibody, for example, may be an antibody that is a phosphotyrosine antibody or a phosphoserine antibody. More particularly, the antibody may be a phospho-EGFR antibody, a phospho-PDGFR antibody, a phospho-KIT antibody, phospho-Bcr-Abl antibody or a phospho-VEGFR antibody. In particular embodiments, the chelator is EC.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The present invention also pertains to imaging agents that comprise a valent metal ion-labeled chelator-antibody conjugate, wherein the antibody is directed against a phosphorylated site of a protein. The antibody can be any of those antibodies discussed above. In some embodiments, the antibody is a phosphotyrosine antibody or a phosphoserine antibody. In particular embodiments, the antibody is an antibody that recognizes a phosphorylated tyrosine residue, such as phospho-EGFR antibody, a phospho-PDGFR antibody, a phospho-KIT antibody, a phospho-Bcr-Abl antibody, a phospho-VEGFR antibody, or a phospho-insulin receptor antibody. The valent metal ion can be any of those valent metal ions discussed above, such as Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1. Summary of receptor tyrosine kinases and cytoplasmic tyrosine kinases.

FIG. 2. Phylogram of the human protein tyrosine kinase family inferred from amino acid sequences of the kinase domains, from Robinson et al., 2000. Numbers on each node indicate the evolutionary distance. The tree is drawn to scale and is midpoint-rooted.

FIG. 3. Summary and classification of human tyrosine kinases.

FIGS. 4A, 4B, 4C, 4D, 4E. Inhibition of phospho-EGFR when cells are exposed to gefitinib treatment. Immunoprecipitation was performed with mouse anti-phosphotyrosine antibody followed by Western blotting to detect the level of phospho-EGFR using anti-EGFR rabbit polyclonal antibody. A. A431 epidermoid carcinoma cells (EGFR amplification); B. H3255 lung adenocarcinoma cells (EGFR mutant); C. MDA-MB-231 breast carcinoma cells (EGFR high expressor); D. H441 lung papillary adenocarcinomas cells (wild-type EGFR); E. Densitometry results of A to D. Results demonstrated a dose-dependent decrease of phospho-EGFR after gefitinib treatment for 6 hours in A431, MDA-MB-231, and H3255 cells but not in H441 cells. The effect was pronounced in H3255 cells (EGFR mutant) and A431 cells (EGFR amplification). No inhibition of phospho-EGFR was observed for H441 cells, even at the highest gefitinib dose level (20 uM). Equal amounts of protein were immunoprecipitated with 2 ug of antibody. The lower band represents heavy-chain IgG.

FIG. 5. Flow cytometry for cell cycle analysis and apoptosis as determined by Annexin-V-Fluos. Cells were cultured for 72 hours with 10 μM of gefitinib then harvested. Apoptosis was quantified by the Annexin-V-Fluos staining followed by FACS analysis. Results are expressed as the percentage of apoptotic cells conferred to the control. Gefitinib-induced apoptosis of H3225, A431, H441, and MDA-MB-231 cells is illustrated. The percentage of apoptotic cells was highest in the H3255 cell line (mutant EGFR-bearing) and the A431 cell line (EGFR amplification).

FIG. 6. High pressure liquid chromatography (HPLC) analysis of ¹¹¹In-EC-P-Tyr. The ultraviolet (UV) (panel A) peak corresponds to sodium iodide radioactive peak (panel B). The concentration used was 10 μg of ¹¹¹In-EC-P-Tyr in 20 μCi. The specific activity was 2 μCi/ug. There were no marked new peaks from ¹¹¹In-EC-P-Tyr suggesting the stability of ¹¹¹In-EC-P-Tyr.

FIG. 7. Planar scintigraphy of ¹¹¹In-EC-Ab in xenograft animal models. The animals received either 2.5% DMSO alone or 100 mg/kg/day gefitinib in 2.5% DMSO for 3 consecutive days, and ¹¹¹In-EC-compound was injected a day after the final treatment. The numbers indicate the T/M uptake 48 hours after injection with the ¹¹¹In-EC-compound. A standard of 27 mCi was placed to help quantify the data. Tumor location is indicated by arrows.

FIGS. 8A, 8B, 8C. Effect of gefitinib on tumor/muscle ratios as determined by imaging with ¹¹¹In-EC-Ab in xenograft animal models. The animals received either 2.5% DMSO alone or 100 mg/kg/day gefitinib in 2.5% DMSO for 3 consecutive days, and then ¹¹¹In-EC-compound was injected at one day after the gefitinib final treatment. The numbers indicate T/M uptake 2, 24, and 48 hours after ¹¹¹In-EC-compound injection. A standard of 27 mCi was placed to help to quantify the data. A. T/M ratios were higher as a function of time with ¹¹¹In-EC-P-Tyr compared to ¹¹¹In-EC-IgG1 at 24 and 48 hours in the untreated A431 group. Decreased T/M ratios were observed by ¹¹¹In-EC-P-Tyr imaging after 3 days geftinib treatment of the A431 xenograft. B. There were no marked changes in T/M ratios between untreated and gefitinib-treated groups in the H441 animal model. C. Region of interest analysis generated from A431 planar images showed that ¹¹¹In-EC-P-Tyr had 40-18% higher T/M ratios than ¹¹¹In-EC-IgG1 in the untreated group (baseline). Decreased T/M ratios (51%-20%) could be measured by 24-48 hours of labelling with ¹¹¹In-EC-P-Tyr but not ¹¹¹In-EC-IgG1 after 3 days of geftinib treatment. The percentage T/M ratio changes between untreated and gefitinib treated cells were minimal in H441 animal models.

FIG. 9. Biodistribution of ¹¹¹In-EC-P-Tyr in human epidermoid cancer cell line (A431) bearing athymic mice (count at 100-475 keV window). % of injected dose per gram of tissue weight (n=3/time interval, iv). Value shown represents the mean±standard deviation of data from 3 animals.

FIG. 10. Biodistribution of ¹¹¹In-EC-IgG1 in human epidermoid cancer cell line (A431) bearing athymic mice (count at 100-475 keV window). % of injected dose per gram of tissue weight (n=3/time interval, iv). Value shown represents the mean±standard deviation of data from 3 animals.

FIG. 11. Biodistribution of ¹¹¹In-EC-P-Tyr tyrosine in human lung papillary carcinoma cell line (H.441) bearing athymic mice (count at 100-475 keV window). % of injected dose per gram of tissue weight (n=3/time interval, iv). Value shown represents the mean±standard deviation of data from 3 animals.

FIG. 12. Biodistribution of ¹¹¹In-EC-IgG1 in human lung papillary carcinoma cell line (H441) bearing athymic mice (count at 100-475 keV window). Value shown represents the mean±standard deviation of data from 3 animals.

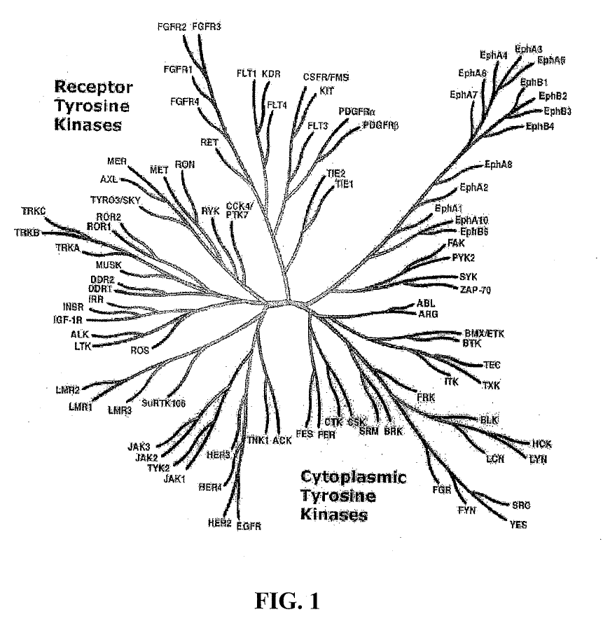

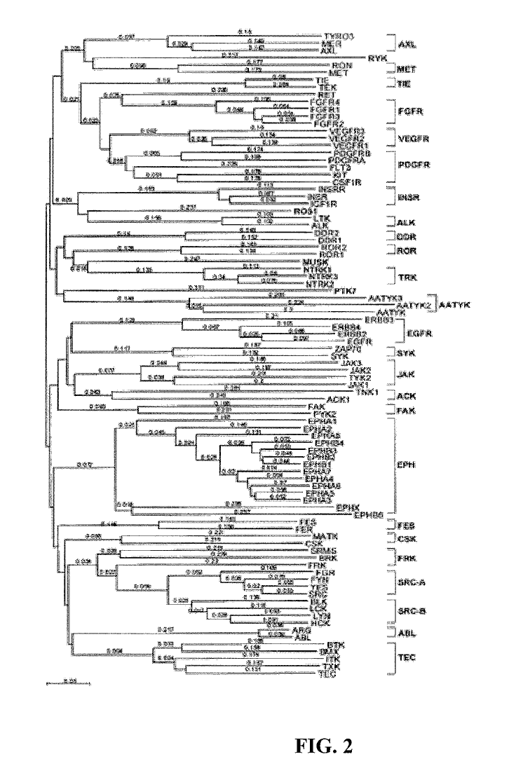

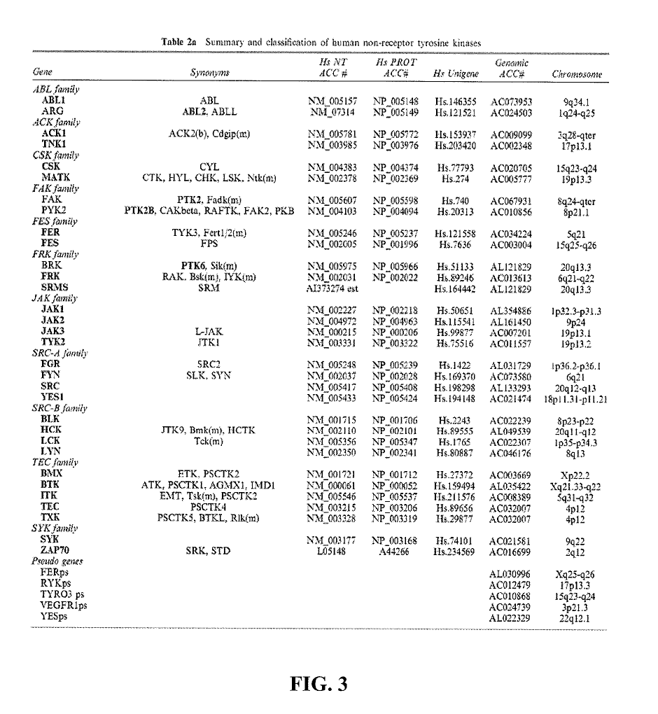

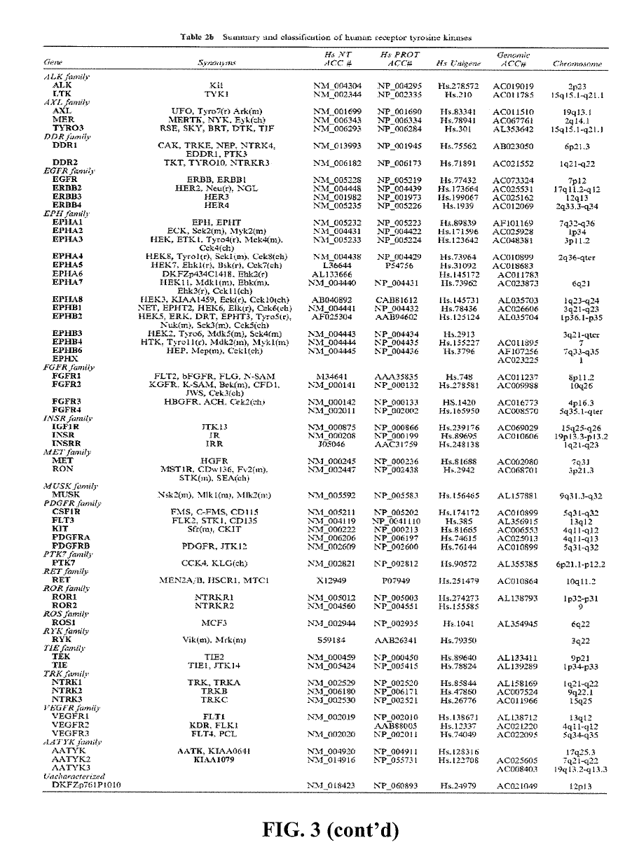

### DESCRIPTION OF ILLUSTRATIVE EMBODIMENTS

### A. Chelators

The chelators that are applied in the compositions and methods set forth herein are capable of binding to an antibody. For example, in particular embodiments, the chelator forms an amide or ester linkage with an amino or carboxyl moiety of the antibody.

Chelation of the valent metal ion to the chelator can be by any method known to those of ordinary skill in the art. Methods of chelation (also called coordination) are described in more detail below. Atoms available for chelation are known to those of skill in the art, and typically comprise O, N or S. In particular embodiments, the atoms available for chelation are selected from the group consisting of N and S.

In some preferred embodiments, the valent metal ion is chelated to a group of atoms selected from the group consisting of NS₂, N₂S, N₄, S₄, N₂S₂, N₃S and NS₃. Chelation can also occur among both the chelator and the antibody—i.e., both the chelator and the antibody may contribute atoms that chelate the same valent metal ion.

In certain embodiments, the chelator is a compound incorporating one or more amino acids. Examples of such amino acids include cysteine and glycine. As discussed below, a linker may connect one amino acid to another. For example, the chelator may comprise three cysteines and one glycine or three glycines and one cysteine. Other examples of such functional groups include hydroxy, thiol, and amido groups.

1. Bis-Aminoethanethiol (BAT) Dicarboxylic Acids

Bis-aminoethanethiol (BAT) dicarboxylic acids may constitute a chelator employed in the method of the present invention. In preferred embodiments, the BAT dicarboxylic acid is ethylenedicysteine (EC). BAT dicarboxylic acids are capable of acting as tetradentate ligands, and are also known as diaminodithiol (DADT) compounds. Such compounds are known to form very stable complexes. The ^{99m}Tc labeled diethylester (^{99m}Tc-L,L-ECD) is known as a brain agent. ^{99m}Tc-L,L-ethylenedicysteine (^{99m}Tc-L,L-EC) is its most polar metabolite and was discovered to be excreted rapidly and efficiently in the urine. Thus, ^{99m}Tc-L,L-EC has been used as a renal function agent. (Verbruggen et al. 1992). Other metals such as indium, rhenium, gallium, copper, holmium, platinum, gadolinium, lutecium, yttrium, cobalt, calcium and arsenic may also be chelated with BAT dicarboxylic acids such as EC.

2. N₄ Chelators

In certain embodiments, the chelator of the present invention comprises an N₄ compound. In some embodiments, the N₄ chelator is cyclic whereas in other embodiments, the N₄ chelator is non-cyclic. Generally, cyclic N₄ chelators are more rigid than their non-cyclic counterparts, and this may be a factor in their efficacy. Certain N₄ compounds are hydrophobic chelators and may be conjugated to other molecules to produce novel compounds which may be used for purposes including imaging and radiotherapy. Certain N₄ compounds may be obtained from commercial sources such as Sigma-Aldrich Chemical Company (Milwaukee, Wis.). U.S. Pat. No. 5,880,281 describes a method for producing certain N₄ compounds.

Non-limiting examples of structures of cyclic N₄ compounds include:

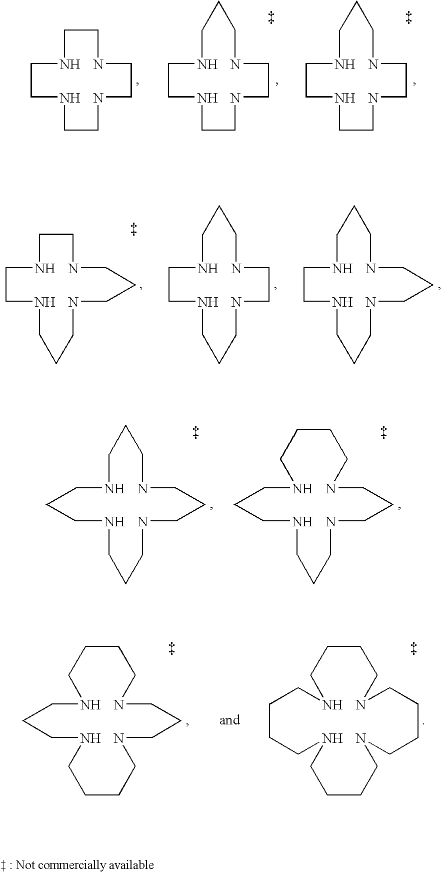

3. Linkers

In some embodiments, the chelator may include two or more moieties joined together by one or more linkers. For example, amino acids and their derivatives may be joined by one or more linkers. An example of two amino acids joined by a linker includes ethylenedicysteine, described above. Such linkers are well known to those of ordinary skill in the art. These linkers, in general, provide additional flexibility to the overall compound that may facilitate chelation of one or more valent metal ions to the chelator. Non-limiting examples of linkers include alkyl groups of any length, such as ethylene (—CH₂—CH₂—), ether linkages, thioether linkages, amine linkages and any combination of one or more of these groups. It is envisioned that multiple chelators (that is, two or more) linked together are capable of forming an overall molecule that may chelate to one or valent metal ions. That is, each chelator that makes up the overall molecule may each chelate to a separate valent metal ion.

### B. Antibodies Directed Against a Phosphorylation Site of a Protein

The term “antibody” is defined herein to refer to a protein or polypeptide produced in a subject in response to a specific antigen which is capable of binding to the antigen. The term “antibody” includes polyclonal antibodies, monoclonal antibodies, chimeric antibodies, humanized antibodies, multispecific antibodies (e.g., bispecific antibodies), as well as fragments, regions or derivatives thereof, provided by any known technique, such as, but not limited to, enzymatic cleavage, peptide synthesis or recombinant techniques.

The antibodies that are used in the compositions and methods of the present invention are antibodies that are directed against a phosphorylation site of a protein. A phosphorylation site of a protein is a moiety that undergoes phosphorylation. For example, the antibody may recognize a phosphorylated tyrosine residue or a phosphorylated serine residue. The antibody may or may not recognize additional sites of the protein that do not undergo phosphorylation, so long as the antibody recognizes at least one phosphorylation site of a protein.

The antibodies directed against a phosphorylation site of a protein of the present invention include at least one of a heavy chain constant region, a heavy chain variable region, a light chain variable region, or a light chain constant region. In some embodiments, a polyclonal antibody, monoclonal antibody, fragment and/or region thereof includes at least one heavy chain variable region or light chain variable region that binds a portion of a phosphorylation site of a protein and/or neutralizes a phosphorylation site of a protein.

A “polyclonal antibody” is defined herein to refer to heterogeneous populations of antibody molecules derived from the sera of animals immunized with an antigen. A “monoclonal antibody” contains a substantially homogeneous population of antibodies specific to antigens, which population contains substantially similar epitope binding sites. The antibodies that are included in the conjugates of the present invention can be prepared by any method known to those of ordinary skill in the art.

For example, a monoclonal antibody may be obtained by methods well-known to those skilled in the art. See, e.g., Kohler and Milstein, 1975; U.S. Pat. No. 4,376,110; Ausubel et al., 1992); Harlow and Lane 1988; Colligan et al., 1993, the contents of which are each herein specifically incorporated by reference. Such antibodies may be of any immunoglobulin class including IgG, IgM, IgE, IgA, and any subclass thereof. A hybridoma producing a mAb of the present invention may be cultivated in vitro, in situ or in vivo.

“Chimeric antibodies” are molecules different portions of which are derived from different animal species, such as those having variable region derived from a murine mAb and a human immunoglobulin constant region, which are primarily used to reduce immunogenicity in application and to increase yields in production. Chimeric antibodies and methods for their production are known in the art. Exemplary methods of production are described in Cabilly et al., 1984; Boulianne et al., 1984; and Neuberger et al., 1985, each of which are herein incorporated by reference in their entirety.

“Humanized” forms of non-human (e.g., murine) antibodies are also contemplated as antibodies in the context of the present invention. Humanized antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al. (1986); Riechmann et al. (1988); and Presta (1992).

“Multispecific antibodies” have binding specificities for at least two different antigens. While such molecules normally will only bind two antigens (i.e., bispecific antibodies, BsAbs), antibodies with additional specificities such as trispecific antibodies are encompassed by this expression when used herein. Examples of BsAbs include those with one arm directed against a phosphorylation site of a protein, and another arm directed against a second antigen that may or may not include a phosphorylation site of a protein. Methods for making bispecific antibodies are known in the art. Traditional production of full-length bispecific antibodies is based on the coexpression of two immunoglobulin heavy chain-light chain pairs, where the two chains have different specificities (see, e.g., Millstein et al., Nature, 305:537-539 (1983)).

### C. Valent Metal Ions

In some embodiments of the compositions of the present invention, the composition further comprises a valent metal ion chelated to the chelator-antibody conjugate. A “valent metal ion” is defined herein to refer to a metal ion that is capable of forming a bond, such as a non-covalent bond, with one or more atoms or molecules. The other atom(s) or molecule(s) may be negatively charged.

Any valent metal ion known to those of ordinary skill in the art is contemplated for inclusion in the compositions of the present invention. One of ordinary skill in the art would be familiar with the valent metal ions and their application(s). In some embodiments, the valent metal ion may be selected from the group consisting of Tc-99m, Cu-60, Cu-61, Cu-62, Cu-67, In-111, Tl-201, Ga-67, Ga-68, As-72, Re-186, Re-188, Ho-166, Y-90, Sm-153, Sr-89, Gd-157, Bi-212, Bi-213, Fe-56, Mn-55, Lu-177, a valent iron ion, a valent arsenic ion, a valent selenium ion, a valent thallium ion, a valent manganese ion, a valent cobalt ion, a valent platinum ion, a valent rhenium ion, a valent calcium ion and a valent rhodium ion. For example, the valent metal ion may be a radionuclide. A radionuclide is an isotope of artificial or natural origin that exhibits radioactivity. In some embodiments, the radionuclide is selected from the group consisting of ^{99m}Tc, ¹⁸⁸Re, ¹⁸⁶Re, ¹⁵³Sm, ¹⁶⁶Ho, ⁹⁰Y, ⁸⁹Sr, ⁶⁷Ga, ⁶⁸Ga, ¹¹¹In, ¹⁸³Gd, ⁵⁹Fe, ²²⁵Ac, ²¹²Bi, ²¹¹At, ⁴⁵Ti, ⁶⁰Cu, ⁶¹Cu, ⁶⁷Cu, ⁶⁴Cu and ⁶²Cu. In preferred embodiments, the valent metal ion is ¹¹¹In or ⁶⁸Ga.

Chelation of a valent metal ion to a chelator-antibody conjugate may require a reducing agent. Any reducing agent known to those of ordinary skill in the art is contemplated. For example, the reducing agent may be a dithionite ion, a stannous ion and a ferrous ion.

A number of factors must be considered for optimal radioimaging in humans. To maximize the efficiency of detection, a valent metal ion that emits gamma energy in the 100 to 200 keV range is preferred. A “gamma emitter” is herein defined as an agent that emits gamma energy of any range. One of ordinary skill in the art would be familiar with the various valent metal ions that are gamma emitters. To minimize the absorbed radiation dose to the patient, the physical half-life of the radionuclide should be as short as the imaging procedure will allow. To allow for examinations to be performed on any day and at any time of the day, it is advantageous to have a source of the radionuclide always available at the clinical site. One of ordinary skill in the art would be familiar with methods to determine optimal radioimaging in humans. Examples are set forth below.

In certain particular embodiments of the present invention, the valent metal ion is a therapeutic valent metal ion. For example, the valent metal ion may be a beta-emitter. As herein defined, a “beta emitter” is any agent that emits beta energy of any range. Examples of beta-emitters include Re-188, Re-186, Ho-166, Y-90, Bi-212, Bi-213, and Sn-153. The beta-emitter may or may not also be gamma-emitter. A “gamma emitter” is any agent that emits gamma energy of any range. One of ordinary skill in the art would be familiar with the use of beta-emitters and gamma emitters in the treatment of a disease, such as cancer.

In further embodiments of the compositions of the present invention, the valent metal ion is a therapeutic valent metal ion that is not a beta emitter or a gamma emitter. For example, the therapeutic metal ion may be platinum, cobalt, copper, arsenic, selenium, calcium or thallium. Compositions including these therapeutic metal ions may be applied in methods directed to the treatment of hyperproliferative disease, such as the treatment of cancer.

In some embodiments, a valent metal ion-labeled chelator-antibody conjugate of the present invention can be applied in performing dual chemotherapy (through chelation to a therapeutic valent metal ion that is not a beta emitter or a gamma emitter) and radiotherapy (through chelation to a valent metal ion that is a beta emitter or a gamma emitter).

### D. Methods of Synthesis

1. Source of Reagents for the Compositions of the Present Invention

Reagents for preparation of the compositions of the present invention can be obtained from any source. A wide range of sources are known to those of ordinary skill in the art. For example, the reagents can be obtained from commercial sources, from chemical synthesis, or from natural sources. The reagents may be isolated and purified using any technique known to those of ordinary skill in the art. Information regarding antibodies and antibody preparation is discussed elsewhere in this specification. Examples of valent metal ions to be employed in the compositions of the present invention include valent metal ions obtained from generators (e.g., Tc-99m, Cu-62, Cu-67, Ga-68, Re-188, Bi-212), cyclotrons (e.g., Cu-60, Cu-61, As-72, Re-186) and commercial sources (e.g., In-111, Tl-201, Ga-67, Y-90, Sm-153, Sr-89, Gd-157, Ho-166).

Methods of preparing and obtaining chelators are well known to those of skill in the art. For example, chelators may be obtained from commercial sources, chemical synthesis, or natural sources.

In one embodiment, the chelator may comprises ethylenedicysteine (EC). The preparation of ethylenedicysteine (EC) is described in U.S. Pat. No. 6,692,724. Briefly, EC may be prepared in a two-step synthesis according to the previously described methods (Ratner and Clarke, 1937; Blondeau et al., 1967; each incorporated herein by reference). The precursor, L-thiazolidine-4-carboxylic acid, was synthesized and then EC was then prepared. It is sometimes also important to include an antioxidant, such as ascorbic acid, in the composition to prevent oxidation of the ethylenedicysteine. Other antioxidants, such as tocopherol, pyridoxine, thiamine, or rutin may also be useful.

Chelators may also comprise amino acids joined together by linkers. Such a linker may comprise, as described above, an alkyl linker such as ethylene.

Amide bonds may also join one or more amino acids together to form a chelator. Examples of synthetic methods for the preparation of such chelators include solid-phase synthesis and solution-phase synthesis. Such methods are described, for example, in Bodansky, 1993 and Grant, 1992.

2. Conjugation of a Chelator to an Antibody Directed Against a Phosphorylation Site of a Protein

Any method known to those of ordinary skill in the art can be used to conjugate a chelator to an antibody directed against a phosphorylation site of a protein. The chelator, for example, may be conjugated to an amino group or a carboxyl group of the antibody to form a chelator-antibody. For example, an amino, carboxyl, or sulfhydryl moiety of a chelator may be conjugated to the antibody. In some embodiments, a carboxyl moiety of a chelator is conjugated to an amino moiety of the antibody.

Most commonly, as between the chelator and the antibody, one acts as the nucleophile and one acts as the electrophile such that conjugation takes place via a covalent bond. Non-limiting examples of such covalent bonds include an amide bond, an ester bond, a thioester bond and a carbon-carbon bond. In preferred embodiments, the conjugation takes place via an amide or ester bond. In some embodiments, the conjugation takes place at one or more functional groups of the chelator selected from the group consisting of carboxylic acid, amine and thiol. When acting as electrophiles, chelators and targeting ligands may comprise functional groups such as halogens and sulfonyls which act as leaving groups during conjugation. Targeting ligands may also comprise nucleophilic groups, such as —NH₂, which may participate in conjugation with an electrophilic chelator. In yet other embodiments, a linker may be used to aid in the conjugation, wherein the linker lies between the chelator and the targeting ligand. Non-limiting examples of such linkers include peptides, glutamic acid, aspartic acid, bromo ethylacetate, ethylene diamine, lysine and any combination of one or more of these groups. Persons of skill in the art will be familiar with these and other types of linkers available for this purpose.

Coupling agents, as used herein, are reagents used to facilitate the coupling of a chelator to a targeting ligand. Such agents are well known to those of ordinary skill in the art and may be employed in certain embodiments of methods of the present invention. Examples of coupling agents include, but are not limited to, sulfo-N-hydroxysuccinimide (sulfo-NHS), dimethylaminopyridine (DMAP), diazabicyclo[5.4.0]undec-7-ene (DBU), 1-ethyl-3-(3-dimethylaminopropyl)carbodiimide (EDAC) and dicyclohexylcarbodiimide (DCC). Other carbodiimides are also envisioned as coupling agents. Coupling agents are discussed, for example, in Bodansky, 1993 and Grant, 1992. These coupling agents may be used singly or in combination with each other or other agents to facilitate conjugation. Once the antibody is conjugated using a coupling agent, urea is typically formed. The urea by-product may be removed by filtration. The conjugated product may then be purified by, for example, silica gel column chromatography or HPLC.

In some embodiments, a linker is used to couple a chelator to an antibody. Examples of linkers include ethylenediamine, amino propanol, diethylenetriamine, aspartic acid, polyaspartic acid, glutamic acid, polyglutamic acid, cysteine, glycine and lysine. For example, U.S. Pat. No. 6,737,247 discloses several linkers which may be used with the present invention and is hereby incorporated by reference in its entirety without disclaimer. U.S. Pat. No. 5,605,672 discloses several “preferred backbones” which may be used as linkers in the present invention and is hereby incorporated by reference in its entirety. In certain embodiments, the chelator may be conjugated to a linker, and the linker is conjugated to the antibody. In other embodiments more than one linker may be used; for example, a chelator may be conjugated to a linker, and the linker is conjugated to a second linker, wherein the second linker is conjugated to the antibody. In certain embodiments, two, three, four, or more linkers that are conjugated together may be used to conjugate a chelator and antibody. However, it is generally preferable to only use a single linker to conjugate a chelator and an antibody.

Some chelators, such as EC, are water soluble. In some embodiments, the chelator-antibody conjugate chelated to a valent metal ion of the invention is water soluble.

Many of the targeting ligands used in conjunction with the present invention will be water soluble, or will form a water soluble compound when conjugated to the chelator. If one reagent is not water soluble, however, a linker which will increase the solubility may be used. Linkers may attach to, for example, an aliphatic or aromatic alcohol, amine, peptide or to a carboxylic acid. Linkers may be, for example, either poly amino acids (peptides) or amino acids such as glutamic acid, aspartic acid or lysine. Table 2 illustrates preferred linkers for specific drug functional groups.

TABLE 2
Linkers

| Drug Functional | | |
|---|---|---|
| Group | Linker | Example |
| Aliphatic or | EC-Poly (glutamic acid) (MW 750- | A |
| phenolic-OH | 15,000) or EC poly(aspartic acid) | |
| | (MW 2000-15,000) or bromo | |
| | ethylacetate or EC-glutamic acid or | |
| | EC-aspartic acid. | |
| Aliphatic or | EC-poly(glutamic acid) (MW 750- | B |
| aromatic-NH₂ or | 15,000) or EC-poly(aspartic acid) | |
| peptide | (MW 2000-15,000) or EC-glutamic | |
| | acid (mono- or diester) or EC-aspartic | |
| | acid. | |
| Carboxylic acid | Ethylene diamine, lysine | C |
| or peptide | | |

3. Chelation of a Valent Metal Ion

The present invention further contemplates methods for the chelation (also called coordination) of one or more valent metal ions to a chelator or a chelator-antibody conjugate. In certain embodiments, the chelator and the antibody may each contribute to the chelation of the valent metal ion. In particular embodiments, the valent metal ion is chelated only to the chelator. The chelated valent metal ion may be bound via, for example, an ionic bond, a covalent bond, or a coordinate covalent bond (also called a dative bond). Methods of such coordination are well known to those of ordinary skill in the art. In one embodiment, coordination may occur by admixing a valent metal ion into a solution containing a chelator. In another embodiment, coordination may occur by admixing a valent metal ion into a solution containing a chelator-antibody conjugate of the present invention. The chelator and the antibody may each be protected by one or more protecting groups before or after chelation with the valent metal ion. For instance, a cyclam, a cyclal, glycine tricysteine peptide or triglycine cysteine peptide could be conjugated to a valent metal ion.

Chelation may occur at any atom or functional group of a chelator or targeting ligand that is available for chelation. The chelation may occur, for example, at one or more N, S, O or P atoms. Non-limiting examples of chelation groups include NS₂, N₂S, N₄, S₄, N₂S₂, N₃S and NS₃, and O₄. In preferred embodiments, a valent metal ion is chelated to three or four atoms. In some embodiments, the chelation occurs among one or more thiol, amine or carboxylic acid functional groups. The chelation, in particular embodiments, may be to a carboxyl moiety of glutamate, aspartate, an analog of glutamate, or an analog of aspartate. These embodiments may include multiple valent metal ions chelated to poly(glutamate) or poly(aspartate) chelators. In some embodiments, chelation of the valent metal ion is to the antibody, such as to carboxyl groups of the antibody.

In general, the reaction is carried out in aqueous media. Any ratio of reagents can be used in the reaction mixture. For example, in some embodiments the ratio of chelator to antibody is 1:1 in aqueous media. In some embodiments of the present methods, a coupling agent is used to couple a chelator to an antibody. In certain embodiments, the coupling agent used in aqueous condition is 1-ethyl-3-(3-dimethylaminopropyl)carbodiimide-HCl (EDC). In some embodiments of the present methods, the chelator is first dissolved in water. An aqueous solution comprising the antibody can then be added to an aqueous solution comprising the chelator. The reaction mixture can then be stirred for 25 hours at room temperature. The product can then be isolated from solution by any method known to those of ordinary skill in the art. For example, the product can be dialyzed from solution using a dialysis membrane that has a cut-off at 1,000 daltons. The product can then be used immediately, or freeze-dried and stored.

Conjugation of the chelator can be to any residue of the antibody. In certain preferred embodiments, the conjugation is to an amino or acid group of the polypeptide.

In some embodiments, a second moiety is conjugated to the chelator-antibody conjugate. For example, the second moiety may be a second antibody, or it may be a therapeutic or tissue-targeting moiety. Therapeutic agents, such as methotrexate or doxorubicin, can be conjugated to amino or acid moieties of the chelator or antibody. Diagnostic agents such as diatrizoic acid, iothalamic acid, and iopanoic acid can be conjugated to amino or acid moieties of the chelator or antibody. Tissue targeting moieties such as hypoxic markers (metronidazole, misonidazole), glycolysis markers (deoxyglucose, glucosamine), amino acids (e.g., tyrosine, lysine), cell cycle markers (e.g., adenosine, guanosine, penciclovir, aminopenciclovir), or receptor markers (e.g., estrogen, folate, androgen) can be conjugated to amino or acid moieties of the antibody or chelator. In particular embodiments, conjugation of a second moiety is to acid moieties of the antibody.

In some embodiments, a diagnostic agent (e.g., x-ray contrast media or optical contrast media) is conjugated to the chelator-antibody conjugate. It may be employed for PET/CT, SPECT/CT, or optical/CT applications. In further embodiments, a radiotherapeutic metallic substance is conjugated to the chelator-antibody conjugate. Such agents may be employed for radiochemotherapy.

4. Purification Procedures and Determinations of Purity

In some embodiments of the methods set forth herein, the chelator-antibody conjugate is purified. Persons of ordinary skill in the art are familiar with methods of purifying compounds of the present invention.

Purification of every compound of the present invention is generally possible, including the purification of intermediates as well as purification of the final products. One of ordinary skill in the art will understand that compounds can generally be purified at any step. Examples of purification methods include gel filtration, size exclusion chromatography (also called gel filtration chromatography, gel permeation chromatography or molecular exclusion), dialysis, distillation, recrystallization, sublimation, derivatization, electrophoresis, silica gel column chromatography and high-performance liquid chromatography (HPLC), including normal-phase HPLC and reverse-phase HPLC. Purification of compounds via silica gel column chromatography or HPLC, for example, offer the benefit of yielding desired compounds in very high purity, often higher than when compounds are purified via other methods. Examples of comparisons of purity of compounds made via organic and wet methodologies and purified by varying methods are provided below.

Methods of determining the purity of compounds are well known to those of skill in the art and include, in non-limiting examples, autoradiography, mass spectroscopy, melting point determination, ultra violet analysis, colorimetric analysis, (HPLC), thin-layer chromatography and nuclear magnetic resonance (NMR) analysis (including, but not limited to, ¹H and ¹³C NMR). In some embodiments, a colorimetric method could be used to titrate the purity of a chelator or chelator-targeting ligand conjugate. For instance, generation of a thiol-benzyl adduct (that is, a thiol functional group protected by a benzyl group) or the performance of an oxidation reaction by using iodine could be used to determine the purity of chelator or chelator-targeting ligand conjugate. In one embodiment, the purity of an unknown compound may be determined by comparing it to a compound of known purity: this comparison may be in the form of a ratio whose measurement describes the purity of the unknown. Software available on varying instruments (e.g., spectrophotometers, HPLCs, NMRs) can aid one of skill in the art in making these determinations, as well as other means known to those of skill in the art.

The free unbound metal ions can be purified with ion-exchange resin or by adding a transchelator (e.g., glucoheptonate, gluconate, glucarate, and acetylacetonate). One of ordinary skill in the art would be familiar with methods of purification, including use of ion-exchange resins and transchelators.

In certain embodiments of the present invention, purification of a compound does not remove all impurities. In some embodiments, such impurities can be identified.

5. Reducing Agents

In certain embodiments, a radiolabeled chelator-antibody conjugate is synthesized by admixing a chelator-antibody conjugate with a radionuclide and a reducing agent to obtain a radionuclide-labeled chelator-antibody conjugate. Examples of reducing agents that can be used include stannous ion in the form of stannous chloride (SnCl₂), dithionate ion, or ferrous ion. It is also contemplated that the reducing agent may be a solid phase reducing agent.

### E. Imaging Modalities

Aspects of the present invention pertain to methods of imaging a site in a subject. Any method of imaging a site in a subject known to those of ordinary skill in the art can be applied in the context of the present invention. For example, nuclear medicine techniques for imaging may be used.

A variety of nuclear medicine techniques for imaging are known to those of ordinary skill in the art.

1. Gamma Camera Imaging

Any of these techniques can be applied in the context of the imaging methods of the present invention. For example, gamma camera imaging is contemplated as a method of imaging that can be utilized for measuring a signal derived from a valent metal ion, such as a radionuclide. One of ordinary skill in the art would be familiar with techniques for application of gamma camera imaging.

2. PET and SPECT

Radionuclide imaging modalities (positron emission tomography, (PET) and single photon emission computed tomography (SPECT)) are diagnostic cross-sectional imaging techniques that map the location and concentration of radionuclide-labeled conjugates.

PET and SPECT provide information pertaining to information at the cellular level, such as cellular viability. In PET, a patient ingests or is injected with a slightly radioactive substance that emits positrons, which can be monitored as the substance moves through the body. Closely related to PET is single-photon emission computed tomography, or SPECT. The major difference between the two is that instead of a positron-emitting substance, SPECT uses a radioactive tracer that emits high-energy photons.

### F. Radiolabeled Agents

As set forth above, certain embodiments of the compositions of the present invention include a valent metal ion chelated to a chelator-antibody conjugate as set forth above, wherein the valent metal ion is a radionuclide. Radiolabeled agents, compounds, and compositions provided by the present invention are provided having a suitable amount of radioactivity. For example, in forming ^{99m}Tc radioactive complexes, it is generally preferred to form radioactive complexes in solutions containing radioactivity at concentrations of from about 0.01 millicurie (mCi) to about 300 mCi per mL.

Radiolabeled imaging agents provided by the present invention can be used for visualizing sites in a mammalian body. In accordance with this invention, the imaging agents are administered by any method known to those of ordinary skill in the art. For example, administration may be in a single unit injectable dose. Any of the common carriers known to those with skill in the art, such as sterile saline solution or plasma, may be utilized after radiolabeling for preparing the compounds of the present invention for injection. Generally, a unit dose to be administered has a radioactivity of about 0.01 mCi to about 300 mCi, preferably 10 mCi to about 200 mCi. The solution to be injected at unit dosage is from about 0.01 mL to about 10 mL.

After intravenous administration of a diagnostically effective amount of a composition of the present invention, imaging can be performed. Imaging of a site within a subject, such as an organ or tumor can take place, if desired, in hours or even longer, after the radiolabeled reagent is introduced into a patient. In most instances, a sufficient amount of the administered dose will accumulate in the area to be imaged within about 0.1 of an hour. As set forth above, imaging may be performed using any method known to those of ordinary skill in the art. Examples include PET, SPECT, and gamma scintigraphy. In gamma scintigraphy, the radiolabel is a gamma-radiation emitting radionuclide and the radiotracer is located using a gamma-radiation detecting camera. The imaged site is detectable because the radiotracer is chosen either to localize at a pathological site (termed positive contrast) or, alternatively, the radiotracer is chosen specifically not to localize at such pathological sites (termed negative contrast).

### G. Kits

Certain embodiments of the present invention are generally concerned with kits for preparing a radiopharmaceutical preparation, wherein the kit includes one or more sealed containers including a predetermined quantity of a chelator-antibody conjugate composition, wherein the antibody is an antibody directed against a phosphorylated site or a protein. Any chelator comprised in a kit of the present invention may optionally be protected by one or more protecting groups.

In some embodiments, the kits of the present invention include one or more sealed vials containing a predetermined quantity of a chelator of the present invention and a sufficient amount of reducing agent to label the chelator with a valent metal ion. In some embodiments of the present invention, the kit includes a valent metal ion that is a radionuclide. In certain further embodiments, the radionuclide is ^{99m}Tc. In further embodiments of the present invention, the chelator is conjugated to an antibody that is directed against a phosphorylated site of a protein. In still further embodiments, the chelator-antibody conjugate is further conjugated a tissue-specific moiety, diagnostic moiety, an imaging moiety, or a therapeutic moiety.

The kit may also contain conventional pharmaceutical adjunct materials such as, for example, pharmaceutically acceptable salts to adjust the osmotic pressure, buffers, preservatives and the like.

In certain embodiments, an antioxidant is included in the composition to prevent oxidation of the chelator moiety. In certain embodiments, the antioxidant is vitamin C (ascorbic acid). However, it is contemplated that any other antioxidant known to those of ordinary skill in the art, such as tocopherol, pyridoxine, thiamine, or rutin, may also be used. The components of the kit may be in liquid, frozen, or dry form. In a preferred embodiment, kit components are provided in lyophilized form.

The cold (that is, non-radioactivity containing) instant kit is considered to be a commercial product. The cold instant kit could serve a radiodiagnostic purpose by adding radionuclide. The technology is known as the “shake and shoot” method to those of skill in the art. The preparation time of radiopharmaceuticals would be less than 15 min. The same kit could also encompass chelators or chelator-antibody conjugates that could be chelated with different metals for different imaging applications. For instance, copper-61 (3.3 hrs half life) for PET; gadolinium for MRI. The cold kit itself could be used for prodrug purposes to treat disease. For example, the kit could be applied in delivery of a therapeutic metal ion to a site in a patient. In these embodiments, the valent metal ion is a therapeutic valent metal ion (e.g., Re-188, Re-186, Ho-166, Y-90, Sr-89, and Sm-153), and the valent metal ion-labeled conjugate can be applied in the treatment or prevention of a disease, such as cancer.

### H. Diseases Associated with Activation of a Kinase

Particular embodiments of the present invention are directed to methods of imaging, diagnosing, or treating a subject, wherein the subject has a disease associated with activation of a kinase. A disease associated with activation of any kinase known to those of ordinary skill in the art is contemplated by the present invention. “Activation of a kinase” is defined herein to refer to an increase in activity of a kinase relative to a control (unaffected) individual or population of individuals who does not have the disease.

For example, the protein that is phosphorylated by the kinase may be a cell surface receptor, such as a growth factor receptor. Examples include EGFR, PDGFR, KIT, Bcr-Abl, VEGFR, and insulin receptor.

Particular examples of diseases associated with activation of a kinase include hyperproliferative disease, an inflammatory disease, a genetic disease, hypereosinophilic disease, a neurodegenerative disease, an autoimmune disease, osteoclast disease, restenosis, hypoinsulinemia, and mast cell disease

A hyperproliferative disease is herein defined as any disease associated with abnormal cell growth or abnormal cell turnover. For example, the hyperproliferative disease may be cancer. The term “cancer” as used herein is defined as an uncontrolled and progressive growth of cells in a tissue. A skilled artisan is aware other synonymous terms exist, such as neoplasm or malignancy or tumor. Any type of cancer is contemplated for treatment by the methods of the present invention. For example, the cancer may be breast cancer, lung cancer, ovarian cancer, brain cancer, liver cancer, cervical cancer, colon cancer, renal cancer, skin cancer, head and neck cancer, bone cancer, esophageal cancer, bladder cancer, uterine cancer, stomach cancer, pancreatic cancer, testicular cancer, lymphoma, or leukemia. In other embodiments of the present invention, the cancer is metastatic cancer.

A neurodegenerative disease is a disease associated with deterioration of neurons that results in abnormal neuronal function and eventual neuronal loss. Examples of such diseases include Alzheimer Disease, Parkinson's Disease, multiple sclerosis, and Creutzfeldt-Jakob disease.

An osteoclast disease is defined herein to refer to any disease associated with abnormal structure of function of osteoclasts. Osteoclasts are cells that are involved in bone resorption. Examples of such diseases include osteoporosis, and Paget's disease of bone.

Mast cell disease is a rare condition caused by an abnormal proliferation of mast cells. Symptoms include itching, abdominal cramping, and anaphylaxis. Mast cells express KIT (CD117), which is the receptor for scf (stem cell factor). In laboratory studies, scf appears to be important for the proliferation of mast cells, and inhibiting KIT with imitinib may reduce the symptoms of mastocytosis.

Examples of inflammatory diseases associated with activation of a kinase include hepatitis, anemia, and chronic thyroiditis.

Autoimmune diseases are also associated with kinase activation. Examples of autoimmune diseases include rheumatoid arthritis, systemic lupus erythematosus, and multiple sclerosis.

Restenosis is also associated with kinase activation. “Restenosis” refers to a reoccurrence of stenosis following a corrective procedure. Restenosis can involve, for example, an artery, such as a coronary artery, where there is a reoccurrence of stenosis following angioplasty or coronary artery bypass grafting. Restenosis can also apply to other arteries and blood vessels, as well as hollow organs following repair of a blockage.

### I. Chemotherapy

In certain embodiments of the present invention, the chelator-antibody conjugates of the present invention are suitable for chemotherapy. For example, the chelator as set forth herein may be chelated to a valent metal ion that is a therapeutic valent metal ion, as discussed above.

In other embodiments, the chelator-antibody conjugate is labeled with a therapeutic valent metal ion. As discussed above, the therapeutic valent metal ion may be chelated to the chelator alone, the antibody alone, or both the chelator and the antibody. Thus, for example, the chelator-antibody conjugate can be applied in targeting treatment to tumor cells that express a cell surface receptor that includes a phosphorylated tyrosine residue or a phosphorylated serine residue.

For example, the valent metal ion may be a beta-emitter. As herein defined, a beta emitter is any agent that emits beta energy of any range. Examples of beta emitters include Re-188, Re-186, Ho-166, Y-90, and Sn-153. One of ordinary skill in the art would be familiar with these agents for use in the treatment of hyperproliferative disease, such as cancer.

One of ordinary skill in the art would be familiar with the design of chemotherapeutic protocols and radiation therapy protocols that can applied in the administration of the compounds of the present invention. As set forth below, these agents may be used in combination with other therapeutic modalities directed at treatment of a hyperproliferative disease, such as cancer. Furthermore, one of ordinary skill in the art would be familiar with selecting an appropriate dose for administration to the subject. The protocol may involve a single dose, or multiple doses. The patient would be monitored for toxicity and response to treatment using protocols familiar to those of ordinary skill in the art.

### J. Pharmaceutical Preparations

In some embodiments, the valent metal ion-labeled chelator-antibody conjugate is in a pharmaceutical composition. Pharmaceutical compositions of the present invention refers to compositions that do not produce an adverse, allergic or other untoward reaction when administered to an animal, such as, for example, a human, as appropriate. Preparation of such compositions are well-known to those of skill in the art in light of the present disclosure. Moreover, for human administration, it will be understood that preparations should meet sterility, pyrogenicity, general safety and purity standards as required by the FDA Office of Biological Standards.

As used herein, “a composition comprising a pharmaceutically effective amount” or “an effective amount of a composition” includes any and all solvents, dispersion media, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, and combinations thereof, as would be known to one of ordinary skill in the art. Except insofar as any conventional carrier is incompatible with the active ingredient, its use in the present compositions is contemplated.

The compositions of the present invention may comprise different types of carriers depending on route of administration, and whether it need to be sterile for such routes of administration as injection. The compositions of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art.

The actual required amount of a composition of the present invention administered to a patient can be determined by physical and physiological factors such as body weight, severity of condition, the tissue to be imaged, the type of disease, previous or concurrent imaging, idiopathy of the patient, and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of the valent metal ion-labeled chelator-antibody conjugate. In other embodiments, the an active compound may comprise between about 2% to about 75% of the weight of the unit, or between about 25% to about 60%, for example, and any range derivable therein. In other non-limiting examples, a dose may also comprise from about 0.1 mg/kg/body weight to about 1000 mg/kg/body weight or any amount within this range, or any amount greater than 1000 mg/kg/body weight per administration.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

The compositions of the present invention may be formulated in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

Sterile injectable solutions may be prepared using techniques such as filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO (dimethylsulfoxide) as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

### K. Combinational Therapy

Certain aspects of the present invention pertain to methods of treating a disease associated with activation of a kinase. As discussed above, the disease may be, for example, cancer.

The valent metal ion-labeled chelator-antibody conjugates can be applied in the treatment of a disease, such as cancer, along with another agent or therapy method, preferably another cancer treatment. Treatment with these compositions of the present invention may precede or follow the other therapy method by intervals ranging from minutes to weeks. In embodiments where another agent is administered, one would generally ensure that a significant period of time did not expire between the time of each delivery, such that the agents would still be able to exert an advantageously combined effect on the cell. For example, it is contemplated that one may administer two, three, three or more doses of one agent substantially simultaneously (i.e., within less than about a minute) with the therapeutic conjugates of the present invention. In other aspects, a therapeutic agent or method may be administered within about 1 minute to about 48 hours or more prior to and/or after administering a therapeutic amount of a chelator-antibody conjugate of the present invention, or prior to and/or after any amount of time not set forth herein. In certain other embodiments, a conjugate of the present invention may be administered within of from about 1 day to about 21 days prior to and/or after administering another therapeutic modality, such as surgery or gene therapy. In some situations, it may be desirable to extend the time period for treatment significantly, however, where several weeks (e.g., about 1 to 8 weeks or more) lapse between the respective administrations.

Various combinations may be employed, as demonstrated below, wherein the claimed agent for dual chemotherapy and radiation therapy is designated “A” and the secondary agent, which can be any other therapeutic agent or method, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the conjugates of the present invention to a patient will follow general protocols for the administration of chemotherapeutics, taking into account the toxicity, if any, of these agents. It also is contemplated that various standard therapies, as well as surgical intervention, may be applied in conjunction with administration of the conjugates of the present invention. Therapies include but are not limited to additional chemotherapy, additional radiotherapy, immunotherapy, gene therapy and surgery.

### L. Examples

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

The following figures, chemical structures and synthetic details provide certain compounds of the present invention.

### Example 1

### Functional Imaging to Assess In Vivo Down-regulation of Phospho-Tyrosine after Gefitinib Treatment of Epidermal Growth Factor Receptor-Bearing Xenografts

### Materials and Methods

Chemicals and Analysis. Sulfo-N-hydroxysuccinimide (sulfo-NHS) and 1-ethyl-3-(3-dimethylaminopropyl)carbodiimide HCl (EDC) were purchased from Pierce Chemical Co. (Rockford, Ill., USA). All other chemicals were purchased from Aldrich Chemical Co. Inc. (Milwaukee, Wis., USA). ¹¹¹In was purchased from DuPont NEN (Boston, Mass., USA).

Antibodies. Phosphotyrosine mouse monoclonal antibody (P-Tyr), used for immunoprecipitation and imaging) and EGFR polyclonal antibody (used for Western blot analysis) were purchased from Cell Signaling Technology Inc. (Danvers, Mass., USA). Mouse IgG1 (Clone, 15H6), an isotypic control for imaging, was purchased from Southern Biotech (Birmingham, Ala., USA). Horseradish peroxidase-conjugated goat anti-rabbit secondary antibody was obtained from Amersham Pharmacia Biotech (Freiburg, Germany).

Cell culture. A431 human epidermoid carcinomas bearing EGFR amplification (Merlino et al., 1985), MDA-MB-231 human breast carcinoma cells (wild-type EGFR, albeit high expressing) (Takabatake et al., 2007), and human lung papillary H441 adenocarcinoma cells (wild-type EGFR) were obtained from American Type Culture Collection (ATCC) (Rockville, Md., USA). The H3255 human lung adenocarcinoma cell line bearing EGFR mutation (Heimberger et al., 2002; Anderson et al., 2001) was a gift from Dr. Matthew Meyerson (Dana-Farber Cancer Institute, Harvard Medical School, Boston, Mass., USA). A431 and MDA-MB-231 cells were cultured in Dulbecco's modified Eagle's medium and Leibovitz's L-15 medium (ATCC) containing 10% heat-inactive fetal bovine serum (FBS) (Invitrogen Corporation, Carlsbad, Calif., USA). RPMI 1640 (Gemini Bio-Products, Woodland, Calif., USA) with 10% FBS was used to maintain the H441 cell line. The H3255 cells were grown in ACL-4 medium (Invitrogen Corporation) with 5% FBS.

Immunoprecipitation and Immunoblotting to Detect the Expression of EGFR Phosphorylation. In a 10-cm² dish, 1×10⁶ cells were incubated at 37° C. in 5% CO₂. After the cells grew to 85% confluence, they were serum starved for 24 hours; treated with 1, 5, 10, 20 μM of gefitinib (AstraZeneca, Wilmington, Del., UK) without serum for 6 hours; and then 20% serum stimulated for 30 minutes. Following treatment with gefitinib at the different concentrations, immunoprecipitation and immunoblotting were performed to determine the levels of EGFR phosphorylation (phospho-EGFR) in the cells. Cells were rinsed twice in ice-cold phosphate-buffered saline (PBS) and scraped into 0.5 mL lysis buffer (Pierce Chemical Co. Rockford, Ill., USA). Lysates were rotated for 10 minutes prior to centrifugation at 14,000 RPM for 10 minutes at 4° C. Determination of total protein was performed using the Bio-Rad protein assay (Bio-Rad Laboratories, Hercules, Calif., USA). Lysates containing 0.5 mg protein were then incubated with the appropriate amount of P-Tyr antibody at 4° C. for 2 hours. Thirty microliters of protein G-agarose beads were then added, and the samples rotated overnight at 4° C. Beads were collected by brief centrifugation, and then washed 3 times, after which the beads were boiled for 5 minutes in the presence of 30 μl of 2× Laemmli sample buffer. Twenty-five microliters of denatured samples were run on 8% sodium dodecyl sulfate-polymerase gels. The gels were then run for 2 hours at room temperature and transferred to a nitrocellulose membrane (Bio-Rad, Hercules, Calif., USA) for 1 hour at 100V and 4° C. After transfer, the membrane was blocked with 0.2% Tris-buffered saline-Tween-20 plus 5% nonfat dry milk for 1 hour at room temperature and probed with the rabbit polyclonal anti-EGFR antibodies at 4° C. overnight. The membrane was washed and then incubated for 1 hour at room temperature with anti-rabbit IgG horseradish peroxidase-conjugated secondary antibody. The membrane was developed using an electrochemiluminescence kit, (Amersham, Little Chalfont, Buckinghamshire, UK) according to the manufacturer's protocol, and then exposed to autoradiographic film and developed.

In vitro Analysis of Apoptosis. Following treatment with 10 μM gefitinib for 72 hours, adherent cells were collected and combined with no adherent cells. The cells were washed in PBS. Cell suspension was then stained with Annexin-V-Fluos (Roche Diagnostics, Mannheim, Germany) for 30 minutes before labeled cells were quantitated by flow cytometry (EpicsXL; Beckman Coulter, MarmiMiami, Fla., USA). Propidium iodide is membrane impermeable and generally excluded from viable cells; it can be used to stain DNA in dead cells. Annexin-V-Fluos can identify both apoptotic and necrotic cells by binding to phosphatidylserine exposed to the outer leaflet of membrane during the apoptotic process (Vermes et al., 1995).

Radiosynthesis of ¹¹¹In-EC-IgG1 and ¹¹¹In-EC-P-Tyr for Functional Imaging. The antibodies were labeled with ¹¹¹In, which has a half-life of 2.805 days. ¹¹¹In-EC-IgG1 was used as a control in which isotopic antibody was attached by a linker EC to the ¹¹¹In label. ¹¹¹In-EC-P-Tyr represents the anti-P-Tyr mouse antibody linked to the ¹¹¹In label. EC was selected as a chelator, because EC drug conjugates could be labeled with ¹¹¹In easily and efficiently with high radiochemical purity and stability (Blondeau et al., 1967; Van Meron et al., 1993; Surma et al., 1994). Synthesis of EC was performed in a two-step manner according to a method previously described (Ilgan et al., 1998; Zareneyrizi et al., 1992). EC was conjugated to IgG1 and P-Tyr antibodies using sulfo-NHS and EDC as coupling agents. Briefly, P-Tyr mouse antibody and isotypic control mouse IgG1 were stirred with EC, sulfo-NHS, and EDC at room temperature for 17 hours. After dialysis, 2.3-3.4 mg of EC-antibody was obtained. ¹¹¹In was added into a vial containing EC antibody to yield ¹¹¹In-EC-IgG1 and ¹¹¹In-EC-P-Tyr. Radiochemical purity for EC antibodies (Rf=0.1) was greater than 95% as determined by using radio-TLC (Bioscan, Inc., Washington, D.C., USA) eluted with saline or acetone. HPLC analysis of ¹¹¹In-EC-P-Tyr was performed to demonstrate specific activity and stability. Bio Sep-SEL-S 3000 (Column 7.8×300 mm) was equipped with two detectors using 0.1% trifluoroacetic acid in water as mobile phase.

Growth of Tumors in Nude Mice after Treatment with Gefitinib. The animal experiments were approved by The University of Texas M. D. Anderson Cancer Center Institutional Animal Care and Use Committee (IACUC). Six- to eight-week-old female nude mice (National Cancer Institute, Bethesda, Md., USA) were inoculated intramuscularly into the hind legs with 0.1 mL of either A431 or H441 tumor-cell suspensions (3×10⁶ cells/mouse) and allowed to form tumors. When tumor sizes reached 1 cm (greatest diameter), the mice were gavaged daily with 100 mg/kg gefitinib dissolved in 2.5% dimethyl sulfoxide (DMSO) (12) or DMSO alone for 3 consecutive days.

Scintigraphic Imaging Studies. Scintigraphic planar imaging studies were used to determine the in vivo tumor-to-muscle (T/M) ratio of P-Tyr activity before and after gefitinib treatment. Animals were divided into 2 groups: group I, control (gavaged with 2.5% DMSO) and group II, treatment (gavaged with 100 mg/kg gefitinib). The antibodies were labeled with ¹¹¹In at a strength of 0.1 mg with 2 mCi/2 mL saline. Group I was subdivided into 2 groups: group IA, ¹¹¹In-EC-IgG1 and group IB, ¹¹¹In-EC-P-Tyr. Group II was also subdivided into 2 groups: group IIA, ¹¹¹In-EC-IgG1 and group IIB, ¹¹¹In-EC-P-Tyr. The imaging studies were performed after 3 consecutive days and during this time, 100 mg/kg gefitinib or DMSO alone was administered orally. Each animal was injected intravenously with 100 uCi of ¹¹¹In-labeled antibody (physical amount 5 μg per mouse) as described above. At 2, 24, and 48 hours following administration of the radiotracers, scintigraphic images were obtained by using a γ-camera (Siemens Medical Solutions, Hoffman, Ill., USA) equipped with a medium energy.

In vivo biodistribution of ¹¹¹In labeled compounds in tumor-bearing mice. Biodistribution studies were used to determine the distribution of labeled anti-phospho-tyrosine mouse antibody to tissue and organs. Six to eight week old female nude mice (NCl, Bethesda, Md.) were inoculated intramuscularly into the hind legs with 0.1 ml of either A431 or H441 tumor cell suspensions (3×10⁶ cells/mouse) and allowed to form tumors until size reached one cm in greatest diameter. Mice were anesthetized with ketamine before each procedure. Separate biodistribution studies using ¹¹¹In-EC-IgG1 (study 1, n=15 mice) and ¹¹¹In-EC-P-Tyr (study 2, n=15 mice) were conducted. For each compound, the animals were divided into five groups for five time intervals (0.5, 2, 4, 24, and 48 hours; n=3/time point). After administration of the radiotracers, the animals were sacrificed and selected tissues were excised, weighed and counted for radioactivity by using a (gamma)-counter (Packard Instruments, Downers Grove, Ill., USA). The biodistribution of tracer in each sample was calculated as percentage of the injected dose per gram of tissue wet weight (% ID/g). Tumor/non-tumor tissue count density ratios were calculated from the corresponding % ID/g.

### Results

Phospho-EGFR Expression is Inhibited in A431, H3255 and MDA-MB-231 Cells Lines but not In the H441 Cell Line. To investigate the effects of gefitinib treatment on the expression of EGFR, phospho-Tyr expression was determined in A431 epidermoid carcinoma, MDA-MB-231 breast carcinoma, H3255 human lung adenocarcinoma, and H441 lung papillary adenocarcinoma cell lines. The cells were treated with different concentrations of gefitinib (1, 5, 10, and 20 μM) and vehicle.

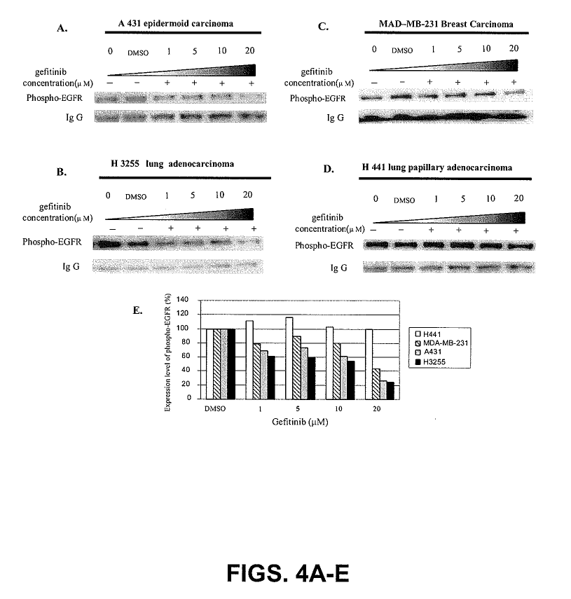

Phospho-EGFR expression was evaluated by Western blot analysis. Phospho-EGFR was inhibited in three cell lines: A431 and MDA-MB-231 (both of which are high expressors of EGFR) and H3255 (mutation-positive EGFR). The MDA-MB-231 cells required 20 μM concentrations to achieve inhibition, whereas in A431 or H3225 cells, only 1 μm of gefitinib achieved inhibition. Phospho-EGFR was not inhibited in the H441 cell line (wild-type EGFR) (FIG. 4A-4D).

Densitometry results demonstrated a dose-dependent decrease of phospho-EGFR after 6-hour gefitinib treatment in A431, MDA-MB-231, and H3255 cells but not in H441 cells (FIG. 4E). The effect was pronounced in H3255 cells and A431 cells. Compared with that in the DMSO control group, inhibition in A431 and H3255 cells was 69.3% and 61.4% (respectively) at 1 μM. MDA-MB-231 attained 58% phospho-EGFR inhibition at the highest concentration (20 μM gefitinib). No phospho-EGFR inhibition was observed in H441 cells, even at the highest dose of gefitinib (20 μM).

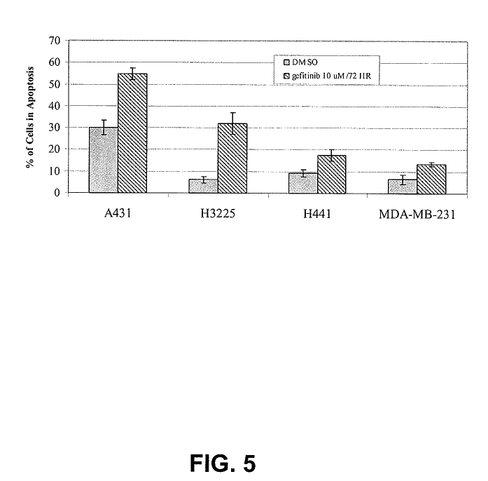

Gefitinib Induces Apoptosis Depending on the Sensitivity of the Cell Line. Results of Annexin-V-Fluos staining followed by fluorescence-activated cell sorting analysis after 72-hours of treatment with 10 μM gefitinib is shown in FIG. 5. Apoptosis was induced differentially in the four cell lines depending on each cell's sensitivity to the drug. (A431 cells also showed baseline apoptosis, probably due to exposure to the vehicle [DMSO]). After deducting the percentage of apoptosis in the DMSO control, the apoptosis percentage was, 25.86% for H3255 and 24.7% for A431. Not surprisingly, phospho-EGFR was not suppressed by 10 μM gefitinib in either H441 or MDA-MB-231 cells. These cells showed low levels of apoptosis (8.13% and 6.92%, respectively) (FIG. 5).

Scintigraphic Imaging of ¹¹¹In-labeled Compounds in an A431 Tumor-bearing Animal Model. Studies were conducted to examine A431 and H441 xenograft models, which are sensitive and resistant, respectively, to EGFR kinase inhibition per our in vitro experiments. H3255 was not examined in vivo, despite its in vitro sensitivity to EGFR kinase inhibition because of the difficulting in creating a xenograft model of this cell line. MDA-MB-231 was not examined because it was not sensitive to in vitro kinase inhibition at levels of exposure to gefitinib below 20 μM. Three animals were used in each experimental group and experiments were repeated twice.

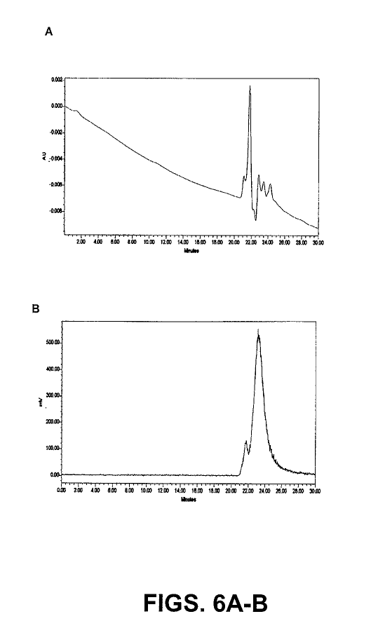

To determine specific activity and stability of our indium-labeled probes, HPLC was used (FIG. 6). The specific activity was 2 μCi/ug. There were no marked new peaks from ¹¹¹In-EC-P-Tyr suggesting the stability of ¹¹¹In-EC-P-Tyr.

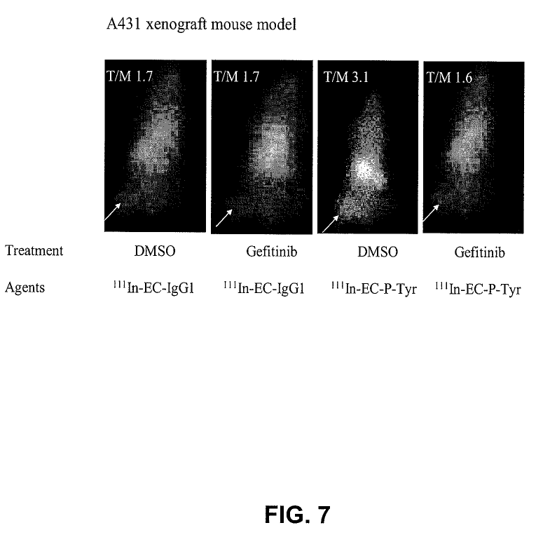

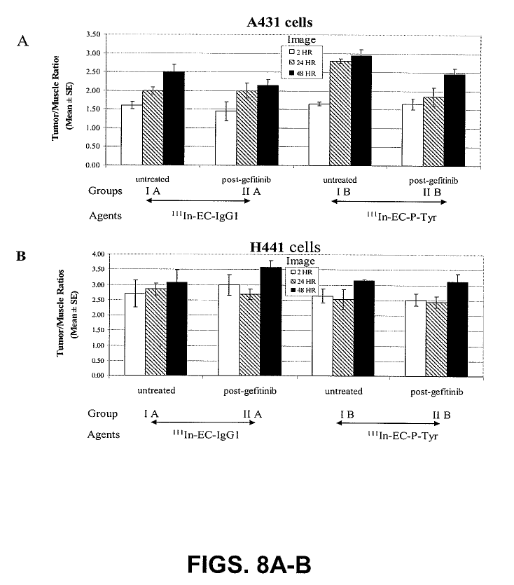

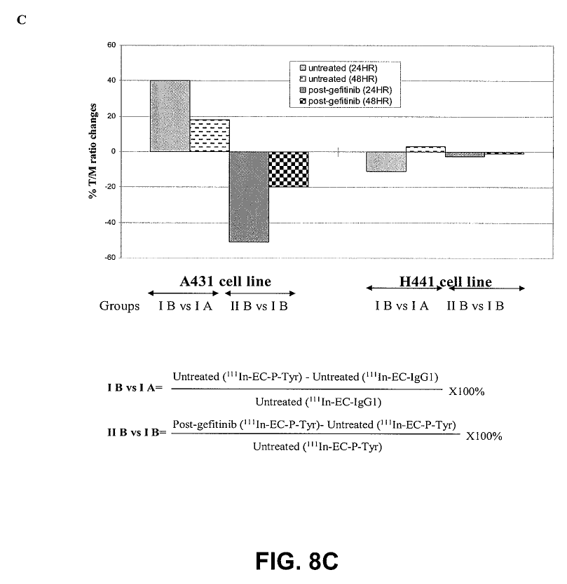

Representative scintigraphic imaging of ¹¹¹In-labeled compounds in A431 tumor-bearing animal models are shown in FIG. 7. The computer-outlined region of interest shows higher T/M ratios as a function of time in ¹¹¹In-EC-P-Tyr compared to ¹¹¹In-EC-IgG1 (control) at 24 and 48 hours (but not at 2 hours after injection of radiolabeled antibody) in the untreated group (FIG. 8A). Decreased T/M ratios were detected by ¹¹¹In-EC-P-Tyr in the geftinib-treated group at 24 and 48 hours with the greatest difference being at 24 hours (FIG. 8A). ¹¹¹ In-EC-P-Tyr produced 18%-40% higher T/M ratios than ¹¹¹In-EC-IgG1 in the untreated group (baseline) (FIG. 8C). Decreased T/M ratios 51%-20% (FIG. 8C) could be measured by using ¹¹¹In-EC-P-Tyr but not by using ¹¹¹In-EC-IgG1 after geftinib treatment (FIG. 8B). This decreased tumor uptake correlated well with the level of expression of phospho-EGFR (inhibited by gefitinib) (shown in FIG. 4). There was no marked change in T/M ratios between untreated and treated groups with gefitinib in H441 tumor-bearing mice (FIGS. 8B, 8C). These findings indicate that gefitinib was able to reduce the level of expression of phospho-EGFR. The physical amount of antibody used was 5 μg/mouse (250 μg/kg).

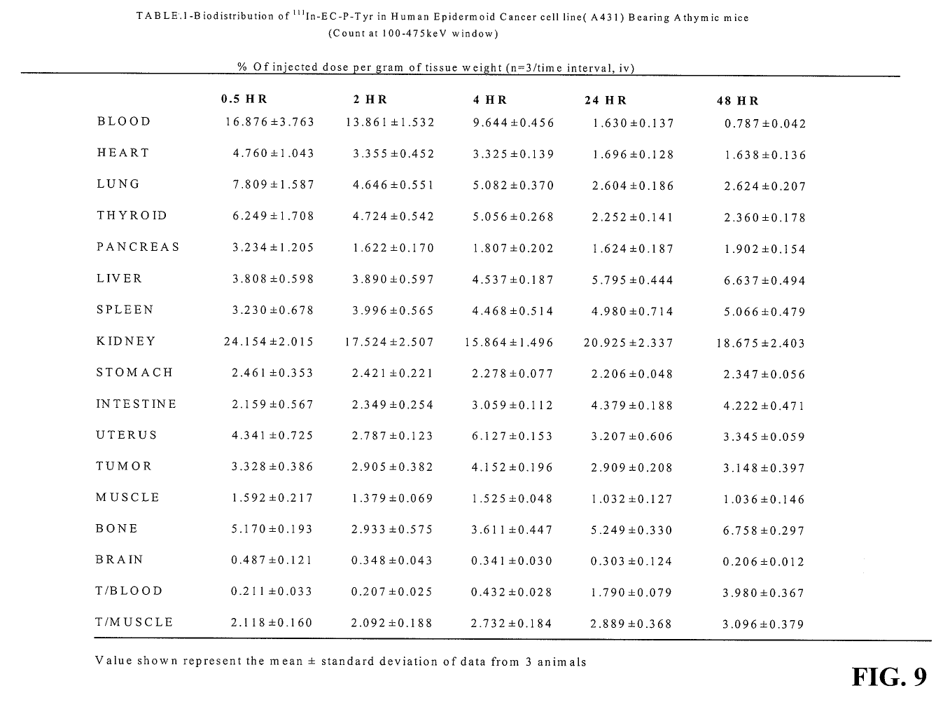

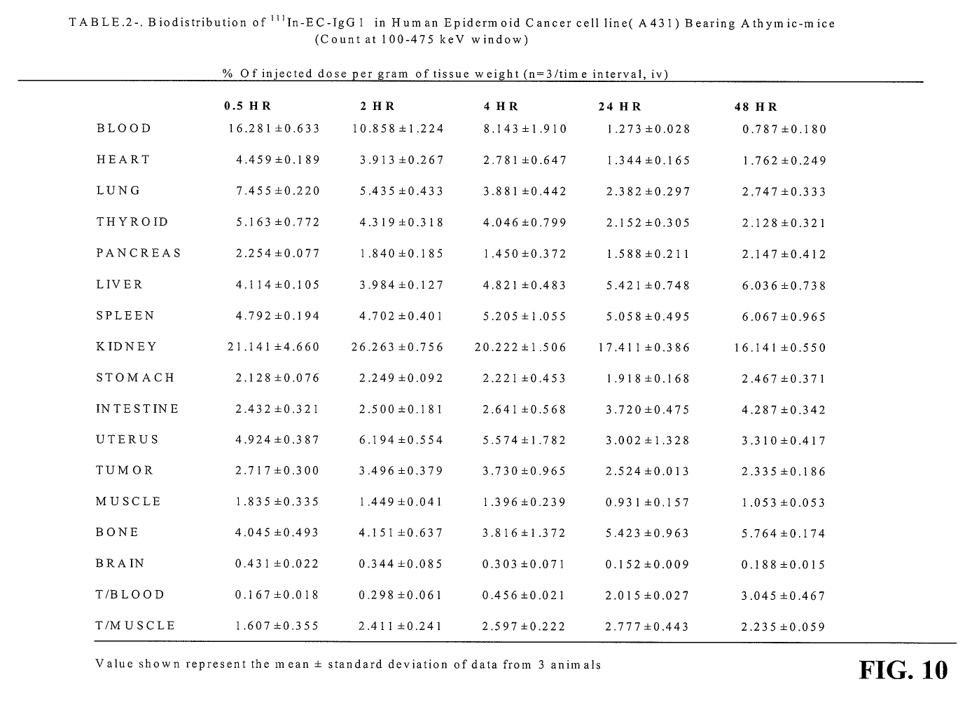

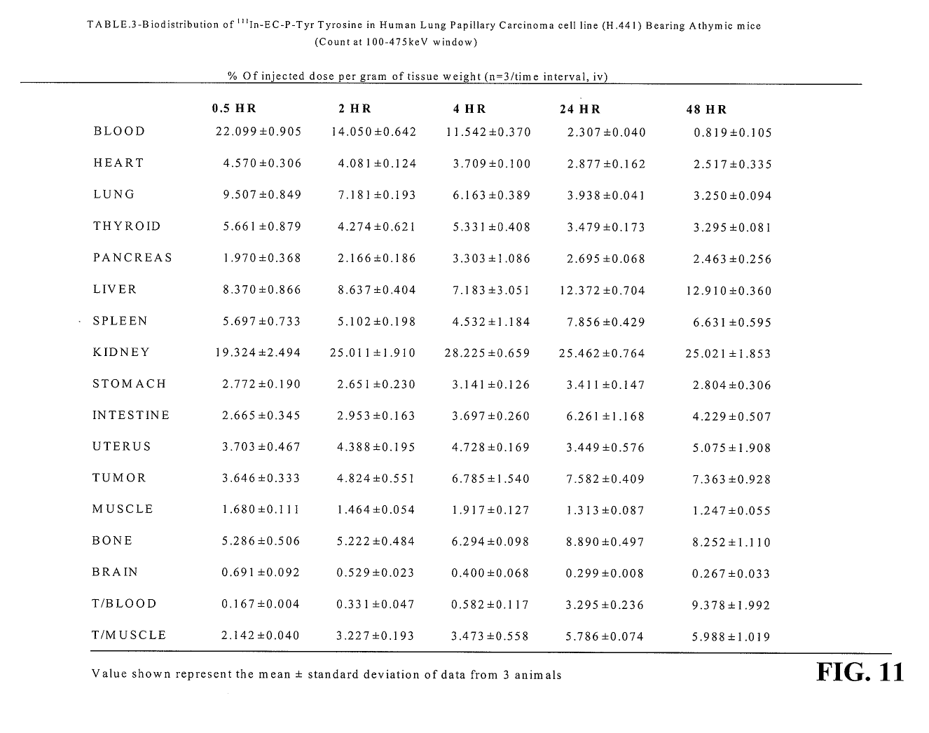

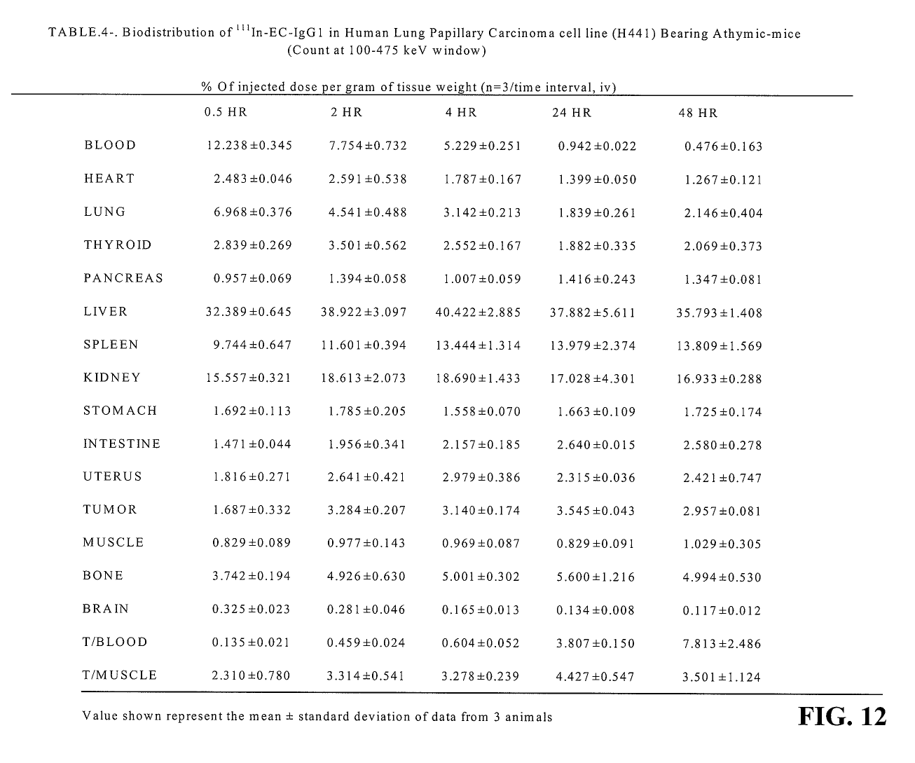

In Vivo biodistribution of ¹¹¹In labeled compounds in tumor-bearing mice. Biodistribution of ¹¹¹In-EC-IgG1 and ¹¹¹In-EC-P-Tyr in tumor-bearing mice (A431 and H441) is shown in FIG. 9-FIG. 12. Biodistribution studies showed that tumor uptake of ¹¹¹In-EC-P-Tyr was significantly higher than control antibody ¹¹¹In-EC-IgG1 (113-140% and 15-35% injected dose/gram) in H441 and A431 animal models. Tumor-to-muscle (T/M) ratios of ¹¹¹In-EC-P-Tyr in A431 and H441 animal models increased as a function of time. At 48 hrs post-administration of radio-tracers, there is 70% increase in T/M ratios of ¹¹¹In-EC-P-Tyr as compared to those of ¹¹¹In-EC-IgG1 in H441 animal models and 39% increase in A431 animal models. These results correlate well to sensitivity of cell lines toward gefitinib treatment. The optimal time of ¹¹¹In-EC-P-Tyr for imaging tumors was at 24-48 hrs.

In summary, these data indicate that radiolabeled antiphosphotyrosine could provide differential diagnosis in drug-sensitive and -resistant models.

### REFERENCES

The following references, to the extent that they provide exemplary procedural or other details supplementary to those set forth herein, are specifically incorporated herein by reference.
U.S. Pat. No. 4,376,110
U.S. Pat. No. 5,605,672
U.S. Pat. No. 5,880,281
U.S. Pat. No. 6,692,724
U.S. Pat. No. 6,737,247
Anderson et al., Int. J. Cancer, 94:774-782, 2001.
Ausubel et al., In: Current Protocols in Molecular Biology, John, Wiley & Sons, Inc, NY, 1992.
Blondeau et al., Can. J. Chem., 45:49-52, 1967.
Bodansky, In: Peptide Chemistry, 2^{nd} ed., Springer-Verlag, New York, 1993.
Boonstra et al., Cell Biol. Int., 19:413-430, 1995.
Boulianne et al., Nature, 312:643-646, 1984.
Cabilly et al., Proc. Natl. Acad. Sci. USA, 91:3273-3277, 1984.
Colligan et al., In: Current Protocols in Immunology, Greene Publ. Assoc. Wiley Interscience, NY, 19931993.
Grant, In: Synthetic Peptides, Freeman & Co., New York, 1992.
Graus-Porta et al., EMBO J., 16:1647-1655, 1997.
Harlow and Lane, In: Antibodies: A Laboratory Manual, Cold Spring Harbor Laboratory, Cold Spring Harbor, N.Y., 346-348, 1988.
Heimberger et al., Clin. Cancer Res., 8:3496-3502, 2002.
Ilgan et al., Cancer Biother. Radiopharm., 13:427-435, 1998.
Jones et al., Nature, 321:522-525, 1986.
Karunagaran et al., EMBO J., 15:254-264, 1996.
Kohler and Milstein, Nature, 256:495-497, 1975.
Laissy et al., Magn. Reson. Imaging., 12:413-419, 1994.
Masui et al., Cancer Res., 44:1002-1007, 1984.
Mendelsohn and Baselga, Oncogene, 19:6550-6565, 2000.
Millstein et al., Nature, 305:537-539, 1983.
Neuberger et al., 1985
Paez et al., Science, 304:1497-1500, 2004.
Park et al., In: Genes, Oncogenes and Hormones. Advances in Cellular and Molecular Biology of Breast Cancer, Dickson and Lippman (Eds.), Boston: Kluiwer Academic Publishers, 194-211, 1991.
Presta, Curr. Op. Struct. Biol., 2:593-596, 1992.
Ratner and Clarke, J. AM Chem. Soc., 59:200-206, 1937.
Riechmann et al., Nature, 332:323-329, 1988.
Robinson et al., Oncogene, 19:5548-5557, 2000.
Sarup et al., Growth Regul., 1:72-82, 1991.
Sato et al., Mol. Biol. Med., 1:511-529, 1983.
Schechter et al., Anticancer Drugs, 14:49-56, 2003.
Song et al., Stroke, 34:982-986, 2003.
Surma et al., Nucl. Med. Commun., 15:628-635, 1994.
Tibes et al., Annu. Rev. Pharmacol. Toxicol., 45:357-84, 2005.
Van Nerom et al., Eur. J. Nucl. Med., 20:738-746, 1993, 1993.
Vermes et al., J. Immunol. Methods, 184:39-51, 1995.
Wu et al., J. Diabetes Complications, 17:297-300, 2003.
Yang and Kim, Eur. J. Nucl. Med. Mol. Imaging, 32:1001-1002, 2005.
Yang et al., Cancer Biother. Radiopharm, 16:73-83, 2001.
Yang et al., Cancer Biother. Radiopharm., 17:233-245, 2002.
Yang et al., Anti-Cancer Drugs, 15:255-263, 2004a.
Yang et al., Cancer Biother. Radiopharm., 19:444-458, 2004b.
Yang et al., Curr. Med, Imaging Rev., 1:25-34, 2005a.
Yang et al., Pharmaceutical Res., 22:1471-1479, 2005b.
Yang et al., Radiology, 226:465-473, 2003.
Zareneyrizi et al., Anticancer Drugs, 10:685-692, 1992.

1. A pharmaceutical composition comprising:
a) a chelator; and
b) an antibody directed against a phosphorylation site of a protein,
wherein the antibody is conjugated to the chelator to form a chelator-antibody conjugate.

2. (canceled)

3. The pharmaceutical composition of claim 1, wherein the protein is a receptor that is a growth factor receptor or a cell surface receptor.

4-5. (canceled)

6. The pharmaceutical composition of claim 1, wherein the antibody is an antibody that recognizes a phosphorylated tyrosine residue or a phosphorylated serine residue.

7. (canceled)

8. The pharmaceutical composition of claim 6, wherein the antibody is a phospho-EGFR antibody, a phospho-PDGFR antibody, a phospho-KIT antibody, a phospho-Bcr-Abl antibody, a phospho-VEGFR antibody, or a phospho-insulin receptor antibody.

9. The pharmaceutical composition of claim 1, wherein the chelator comprises three or more atoms, wherein each atom is selected from the group consisting of nitrogen, sulfur, oxygen, and phosphorus.

10. (canceled)

11. The pharmaceutical composition of claim 9, wherein the chelator is an N₂S₂ chelator.

12. (canceled)

13. The pharmaceutical composition of claim 11, wherein the chelator is N,N-ethylenedicysteine.

14. The pharmaceutical composition of claim 1, wherein the chelator is conjugated to the amino terminus of the antibody or a lysine residue of the antibody.

15. The pharmaceutical composition of claim 1, further comprising a valent metal ion chelated to said chelator-antibody conjugate.

16. (canceled)

17. The pharmaceutical composition of claim 15, wherein the valent metal ion is a radionuclide selected from the group consisting of Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90.

18. The pharmaceutical composition of claim 17, wherein the radionuclide is In-111.

19. (canceled)

20. The pharmaceutical composition of claim 17, comprising In-111 and Y-90.

21. The pharmaceutical composition of claim 1, wherein the chelator is N,N-ethylenedicysteine and wherein the antibody is an phosphotyrosine antibody.

22. A method of synthesizing a radiolabeled chelator-antibody conjugate comprising:
a) obtaining an antibody directed against a phosphorylation site of a protein;
b) admixing said antibody with a chelator to obtain a chelator-antibody conjugate; and
c) admixing said chelator-antibody conjugate with a radionuclide to obtain a radionuclide labeled chelator-antibody conjugate.

23. (canceled)

24. The method of claim 22, wherein the protein is a receptor that is a growth factor receptor or a cell surface receptor.

25-26. (canceled)

27. The method of claim 22, wherein the antibody is an antibody that recognizes a phosphorylated tyrosine residue or a phosphorylated serine residue.

28. (canceled)

29. The method of claim 27, wherein the antibody is a phospho-EGFR antibody, a phospho-PDGFR antibody, a phospho-KIT antibody, a phospho-Bcr-Abl antibody, a phospho-VEGFR antibody, or a phospho-insulin receptor antibody.

30. The method of claim 22, wherein the chelator comprises three or more atoms, wherein each atom is selected from the group consisting of nitrogen, sulfur, oxygen, and phosphorus.

31-33. (canceled)

34. The method of claim 30, wherein the chelator is N,N-ethylenedicysteine.

35-37. (canceled)

38. The method of claim 22, wherein said radionuclide is selected from the group consisting of Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90.

39. The method of claim 38, wherein the radionuclide is In-111.

40. The method of claim 22, further defined as comprising admixing said chelator-antibody conjugate with a radionuclide and a reducing agent.

41. A method for imaging a site in a subject, comprising:
a) administering to the subject an effective amount of a first composition comprising a valent metal ion-labeled chelator-antibody conjugate, wherein the antibody is an antibody directed against a phosphorylation site of a protein; and
b) detecting a radioactive signal from the site in the subject following administration of an effective amount of the first composition.

42. The method of claim 41, wherein the subject is a human.

43-44. (canceled)

45. The method of claim 41, wherein the subject has a disease selected from the group consisting of cancer, an inflammatory disease, a genetic disease, an autoimmune disease, hypereosinophilic syndrome, anemia, osteoclast disease, restenosis, diabetes, and mast cell disease.

46. The method of claim 45, wherein the disease is cancer.

47. The method of claim 46, wherein the cancer is breast cancer, lung cancer, prostate cancer, ovarian cancer, brain cancer, liver cancer, cervical cancer, colon cancer, renal cancer, skin cancer, head and neck cancer, bone cancer, esophageal cancer, bladder cancer, uterine cancer, lymphatic cancer, stomach cancer, pancreatic cancer, testicular cancer, leukemia, lymphoma, or sarcoma.

48. The method of claim 46, wherein the cancer is a metastatic cancer.

49. The method of claim 45, wherein the disease is an inflammatory disease that is hepatitis or chronic thyroiditis.

50. The method of claim 45, wherein the disease is an autoimmune disease that is rheumatoid arthritis, systemic lupus erythematosus, or multiple sclerosis.

51-53. (canceled)

54. The method of claim 41, wherein the protein is a receptor that is a growth factor receptor or a cell surface receptor.

55-56. (canceled)

57. The method of claim 41, wherein the antibody is an antibody that recognizes a phosphorylated tyrosine residue or a phosphorylated serine residue.

58. (canceled)

59. The method of claim 57, wherein the antibody is a phospho-EGFR antibody, a phospho-PDGFR antibody, a phospho-KIT antibody, a phospho-Bcr-Abl antibody, a phospho-VEGFR antibody, or a phospho-insulin receptor antibody.

60. The method of claim 41, wherein the chelator comprises three or more atoms, wherein each atom is selected from the group consisting of nitrogen, sulfur, oxygen, and phosphorus.

61-63. (canceled)

64. The method of claim 60, wherein the chelator is N,N-ethylenedicysteine.

65. The method of claim 41, wherein the chelator is conjugated to the amino terminus of the antibody or a lysine residue of the antibody.

66. (canceled)

67. The method of claim 41, wherein said valent metal ion is a radionuclide selected from the group consisting of Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90.

68. (canceled)

69. The method of claim 41, wherein the valent metal ion-labeled chelator-antibody is further defined as comprising two or more valent metal ions chelated to said chelator-antibody conjugate.

70. The method of claim 69, wherein the valent metal ions are selected from the group consisting of In-111 and Y-90.

71. The method of claim 41, wherein the chelator is N,N-ethylenedicysteine and wherein the antibody is an phosphotyrosine antibody.

72. The method of claim 41, wherein administering comprises intravenous, intracardiac, intradermal, intralesional, intrathecal, intracranial, intrapericardial, intraumbilical, intraocular, intraarterial, intraperitoneal, intratumor, subcutaneous, intramuscular, or intravitreous administration.

73. The method of claim 41, wherein the signal is detected using a signal selected from the group consisting of PET, CT, SPECT, MRI, optical imaging and ultrasound.

74-76. (canceled)

77. The method of claim 41, wherein the site in the subject is a tumor.

78-79. (canceled)

80. The method of claim 77, further comprising treating the subject with phosphotyrosine therapy after steps (a) and (b), and then repeating steps (a) and (b), wherein the radioactive signal diminishes in size or intensity following treatment, wherein the phosphotyrosine therapy is gefitinib, imatinib mesylate, HER-2 antibody, tiludronate, a PDGFR inhibitor, or a glucocorticoid.

81-86. (canceled)

87. The method of claim 41, wherein the first composition comprises more than one valent metal ion, wherein each valent metal ion is selected from the group consisting of Tc-99m, Cu-60, Cu-61, Cu-62, Cu-67, In-111, Tl-201, Ga-67, Ga-68, As-72, Re-186, Re-187, Re-188, Ho-166, Y-90, Sm-153, Sr-89, Gd-157, Bi-212, Bi-213, and Y-90.

88. The method of claim 87, wherein the composition comprises Y-90 and In-111.

89. A kit for preparing a radiopharmaceutical preparation, said kit comprising one or more sealed containers, and a predetermined quantity of a chelator-antibody conjugate composition of claim 1, wherein said antibody is an antibody directed against a phosphorylated site of a protein.

90. (canceled)

91. The kit of claim 89, wherein the protein is a receptor that is a growth factor receptor or a cell surface receptor.

92-93. (canceled)

94. The method of claim 89, wherein the antibody is an antibody that recognizes a phosphorylated tyrosine residue or a phosphorylated serine residue.

95. (canceled)

96. The kit of claim 94, wherein the antibody is a phospho-EGFR antibody.

97. A reagent for preparing a scintigraphic imaging agent comprising an antibody directed against a phosphorylated site of a protein, wherein the antibody is covalently linked to a chelator.

98. The reagent of claim 97, wherein the antibody is a phosphotyrosine antibody or a phosphoserine antibody.

99. (canceled)

100. The reagent of claim 97, wherein the protein is a receptor that is a growth factor receptor or a cell surface receptor.

101-102. (canceled)

103. The reagent of claim 97, wherein the antibody is an antibody that recognizes a phosphorylated tyrosine residue or a phosphorylated serine residue.

104. (canceled)

105. The reagent of claim 103, wherein the antibody is a phospho-EGFR antibody, a phospho-PDGFR antibody, a phospho-KIT antibody, a phospho-Bcr-Abl antibody, a phospho-VEGFR antibody, or a phospho-insulin receptor antibody.

106-112. (canceled)
